SEQUENCE LISTING (17 sequences)

SEQ ID NO: 1 - protein, 21 aa, Artificial sequence
  Synthetic construct
  MISC_FEATURE: Signal Sequence
MALPVTALLLPLALLLHAARP

SEQ ID NO: 2 - protein, 10 aa, Artificial sequence
  Synthetic construct
  MISC_FEATURE: Myc Tag
EQKLISEEDL

SEQ ID NO: 3 - protein, 128 aa, Artificial sequence
  Synthetic construct
  MISC_FEATURE: Lag_17
MADVQLVESGGGLVQAGGSLRLSCAASGRTISMAAMSWFRQAPGKEREFVAGISRSAGSAVHADSVKGRFTISRDNTKNTLYLQMNSLKAEDTAVYYCAVRTSGFFGSIPRTGTAFDYWGQGTQVTVS

SEQ ID NO: 4 - protein, 326 aa, Artificial sequence
  Synthetic construct
  MISC_FEATURE: Notch_Core
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLHLMYVAAAAFVLLFFVGCGVLLSRKRRR

SEQ ID NO: 5 - protein, 112 aa, Artificial sequence
  Synthetic construct
  MISC_FEATURE: CD3-Zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 42 aa, Artificial sequence
  Synthetic construct
  MISC_FEATURE: 4-1BB co-stimulatory domain
KRGRKKLLYIFKQPFMRPVQTTQEEDGCSCRFPEEEEGGCEL

SEQ ID NO: 7 - protein, 41 aa, Artificial sequence
  Synthetic construct
  MISC_FEATURE: CD28 co-stimulatory domain
RSKRSRLLHSDYMNMTPRRPGPTRKHYQPYAPPRDFAAYRS

SEQ ID NO: 8 - protein, 211 aa, Artificial sequence
  Synthetic construct
  MISC_FEATURE: Gal4-VP64
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSAAAGGSGGSGGSDALDDFDLDMLGSDALDDFDLDMLGSDALDDFDLDMLGSDALDDFDLDMLGS

SEQ ID NO: 9 - protein, 8 aa, Artificial sequence
  Synthetic construct
  MISC_FEATURE: GS Linker
GSGSGSGS

SEQ ID NO: 10 - protein, 242 aa, Artificial sequence
  Synthetic construct
  MISC_FEATURE: CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 11 - protein, 299 aa, Artificial sequence
  Synthetic construct
  MISC_FEATURE: Notch_Core_Linking_polypeptide
ILDYSFTGGAGRDIPPPQIEEACELPECQVDAGNKVCNLQCNNHACGWDGGDCSLNFNDPWKNCTQSLQCWKYFSDGHCDSQCNSAGCLFDGFDCQLTEGQCNPLYDQYCKDHFSDGHCDQGCNSAECEWDGLDCAEHVPERLAAGTLVLVVLLPPDQLRNNSFHFLRELSHVLHTNVVFKRDAQGQQMIFPYYGHEEELRKHPIKRSTVGWATSSLLPGTSGGRQRRELDPMDIRGSIVYLEIDNRQCVQSSSQCFQSATDVAAFLGALASLGSLNIPYKIEAVKSEPVEPPLPSQLH

SEQ ID NO: 12 - protein, 21 aa, Artificial sequence
  Synthetic construct
  MISC_FEATURE: Notch_Core_TMD
LMYVAAAAFVLLFFVGCGVLL

SEQ ID NO: 13 - protein, 4 aa, Artificial sequence
  Synthetic construct
  MISC_FEATURE: Cleavage sequence of MMP-9
  misc_feature (2)..(3): Xaa can be any naturally occurring amino acid
  MISC_FEATURE (4)..(4): Xaa is Leu, Ile, Val, Phe, Trp, Tyr, Val, Met, or Pro
PXXX

SEQ ID NO: 14 - protein, 5 aa, Artificial sequence
  Synthetic construct
  MISC_FEATURE: Cleavage sequence of MMP-9
  misc_feature (2)..(3): Xaa can be any naturally occurring amino acid
  MISC_FEATURE (4)..(4): Xaa is Leu, Ile, Val, Phe, Trp, Tyr, Val, Met, or Pro
  MISC_FEATURE (5)..(5): Xaa is Ser or Thr
PXXXX

SEQ ID NO: 15 - protein, 6 aa, Artificial sequence
  Synthetic construct
  MISC_FEATURE: Cleavage sequence of MMP-9
  MISC_FEATURE (2)..(2): Xaa is Leu or Gln
PXGMTS

SEQ ID NO: 16 - protein, 5 aa, Artificial sequence
  Synthetic construct
  MISC_FEATURE: Cleavage sequence of MMP-9
  MISC_FEATURE (2)..(2): Xaa is Leu or Gln
PXGMT

SEQ ID NO: 17 - protein, 7 aa, Artificial sequence
  Synthetic construct
  MISC_FEATURE: Cleavage sequence of MMP-9
ENLYTQS

(19) United States
(12) Patent Application Publication
(10) Pub. No.: US 2024/0299545 A1
(43) Pub. Date: Sep. 12, 2024

(54) HYBRID RECEPTORS WITH MULTIPLE TRANSCRIPTIONAL REGULATORS

(71) Applicant: The Regents of the University of California, Oakland, CA (US)

(72) Inventors: Kole T. ROYBAL, San Francisco, CA (US); Wendell A. LIM, San Francisco, CA (US); Joseph H. CHOE, San Francisco, CA (US)

(21) Appl. No.: 18/552,131

(22) PCT Filed: Mar. 23, 2022
(86) PCT No.: PCT/US2022/021588

§ 371 Date: Sep. 22, 2023

Related U.S. Application Data
(60) Provisional application No. 63/165,442, filed on Mar. 24, 2021.

(51) Int. Cl. A61K 39/00; A61K 38/00; A61K 45/06; A61P 35/00; C07K 14/705; C07K 14/725; C07K 16/28; C07K 16/30; C12N 5/0783

(52) U.S. Cl. CPC A61K 39/4631; A61K 39/464404; A61K 39/464406; A61K 39/464412; A61K 39/464417; A61K 39/464424; A61K 39/464429; A61K 39/464482; A61K 45/06; A61P 35/00; C07K 14/705; C07K 14/70503; C07K 14/70507; C07K 14/7051; C07K 14/70521; C07K 14/70578; C07K 16/2803; C07K 16/2863; C07K 16/2878; C07K 16/2887; C07K 16/2896; C07K 16/30; C07K 16/3007; C12N 5/0636; A61K 38/00; A61K 2039/545; C07K 2319/02; C07K 2319/03; C07K 2319/30; C12N 2510/00

(57) ABSTRACT

The present disclosure relates generally to the field of immunology, and particularly relates to hybrid chimeric antigen receptors designed to combine fast time-scale intracellular signal transduction and long time-scale transcription regulation. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### CROSS REFERENCE TO RELATED APPLICATION

The present application claims priority to U.S. Provisional Patent Application Ser. No. 63/165,442 filed on Mar. 24, 2021, the disclosure of which is incorporated by reference herein in its entirety, including any drawings.

### STATEMENT REGARDING FEDERALLY SPONSORED R&D

This invention was made with government support under grant nos. OD025751, RO1 CA196277 and U54 CA244438 awarded by The National Institutes of Health. The government has certain rights in the invention.

### INCORPORATION OF THE SEQUENCE LISTING

This application contains a Sequence Listing, which is hereby incorporated herein by reference in its entirety. The accompanying Sequence Listing text file, named “048536-695001WO_Sequence Listing_ST25.txt” was created on Mar. 18, 2022 and is 16 KB.

### FIELD

The present disclosure relates generally to the field of immunology, and particularly relates to hybrid chimeric antigen receptors designed to combine fast time-scale intracellular signal transduction and long time-scale transcription regulation. The disclosure also provides compositions and methods useful for producing such receptors, nucleic acids encoding same, host cells genetically modified with the nucleic acids, as well as methods for modulating an activity of a cell and/or for the treatment of various health conditions or diseases, such as cancers.

### BACKGROUND

Notch receptors are transmembrane proteins that mediate cell-cell contact signaling and play a central role in development and other aspects of cell-to-cell communication. Notch receptors are involved in and are required for a variety of cellular functions during development, and are important for the function of a vast number of cell-types across species.

A number of existing synthetic derivatives of Notch receptors, which are often referred to as “SynNotch” have been developed recently by replacing the extracellular ligand-binding domain, which in wild-type Notch contains multiple EGF-like repeats, with an antibody derivative, and replacing the cytoplasmic domain with a transcription activator of choice, but still relying on the Notch NRR (L. Morsut et al., Cell (2016) 164:780-91) and the standard two-step proteolysis. However, the NRR spans approximately 160 amino acids, making this domain alone about three times the size of some mature proteins, such as insulin or epidermal growth factor (EGF). This makes expression of the receptor less efficient, and can exceed the capacity of some widely used cloning and transfection vectors.

In addition, these SynNotch receptors, in contrast to chimeric antigen receptor (CARs), do not elicit membrane proximal signaling via kinase cascades. The receptors, instead, convert ligand binding to a release of a receptor tethered transcription factor that shuttles to the nucleus to regulate a user-defined transcriptional circuit. In particular, these SynNotch receptors lack the ability to initiate fast timescale signaling that regulates cellular processes such as metabolic reprogramming, proliferation, growth factor production, or cytotoxicity.

The present disclosure provides, among other things, a new class of hybrid SynNotch receptors that incorporate intracellular signaling domains (e.g., stimulatory and co-stimulatory domains from, for example, 4-1BB, CD28, and CD3zeta, etc.) that can initiate activation of T cells concomitant with custom transcriptional regulation.

### SUMMARY

Provided herein, among others, is a chimeric receptor comprising, from N-terminus to C-terminus: (a) an extracellular ligand-binding domain having a binding affinity for a selected ligand; (b) a linking polypeptide comprising a sequence that is at least about 80% sequence identity to a Notch juxtamembrane domain (JMD); (c) a transmembrane domain comprising one or more ligand-inducible proteolytic cleavage sites; and (d) an intracellular domain. In some embodiments, the intracellular domain comprises, in any order: (i) an intracellular signaling domain (SD) comprising at least one activation domain, and (ii) a transcriptional regulator. In some embodiments, binding of the selected ligand to the extracellular ligand-binding domain induces cleavage at the ligand-inducible proteolytic cleavage site disposed between the transcriptional regulator and the linking polypeptide.

In some embodiments, the extracellular domain comprises an antigen-binding moiety capable of binding to a ligand on the surface of a cell. In certain embodiments, the cell is a pathogen. In certain embodiments, the cell is a human cell. In certain embodiments, the human cell is a tumor cell. In some embodiments, the human cell is a terminally differentiated cell.

In some embodiments, the ligand comprises a protein or a carbohydrate. In some embodiments, the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), blue fluorescent protein (BFP) enhanced green fluorescent protein (EGFP), and signal regulatory protein α (SIRPα).

In some embodiments, the ligand is selected from cell surface receptors, adhesion proteins, integrins, mucins, lectins, tumor associated antigens, and tumor-specific antigens. In other embodiments, the ligand is a tumor-associated antigen or a tumor-specific antigen.

In some embodiments, the extracellular ligand-binding domain comprises the ligand-binding portion of a receptor. In some embodiments, the antigen-binding moiety is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment thereof. In certain embodiments, the antigen-binding moiety comprises a scFv. In some embodiments, the antigen-binding moiety specifically binds to a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD117), MUC1, NCAM, PAP, PDGFR-0, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

In some embodiments, the tumor-associated antigen is CD19, BCMA, CEA, HER2, MUC1, CD20, ALPPL2, SIRPα, or EGFR. In certain embodiments, the tumor-associated antigen is CD19, BCMA, HER2, or ALPPL2.

In some embodiments, the linking polypeptide comprises a sequence that is at least about 85%, at least about 90%, or at least about 95% identical to a Notch juxtamembrane domain (JMD). In some embodiments, the linking polypeptide comprises one or more LIN-12-Notch repeat (LNR) of a Notch JMD. In some embodiments, the linking polypeptide further comprises one or more heterodimerization domains (HD) of a Notch JMD. In some exemplary embodiments, the linking polypeptide comprises an amino acid sequence having at least 80% sequence identity to SEQ ID NO: 11. In other exemplary embodiments, the transmembrane domain comprises an amino acid sequence having at least 80% sequence identity to SEQ ID NO: 12.

In some embodiments, the intracellular SD further comprises at least one costimulatory domain, each derived from a signaling molecule. In some embodiments, the signaling molecule comprises a class 1 or a class 3 human membrane protein. In certain embodiments, the signaling molecule is selected from the group consisting of CD28, 4-1BB, OX40, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-PlA, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC. In some exemplary embodiments, the signaling molecule is selected from the list consisting of OX40, ICOS, 4-1BB, CTLA4, CD28, CD30, CD2, CD27, and CD226.

In some embodiments, the activation domain comprises one or more immunoreceptor tyrosine-based activation motifs (ITAMs). In some embodiments, the one or more ITAMs are derived from CD3ζ, CD3σ, CD3/, and CD3ε. In some embodiments, the one or more ITAMs are at least about 80, 85, 90, 95, 96, 97, 98, 99, or 100% identical to a CD3ζ ITAM.

In some embodiments, the transcriptional regulator comprises a transcriptional activator, or a transcriptional repressor. In some embodiments, the intracellular domain comprises a nuclear localization sequence and a transcriptional regulator sequence selected from the group consisting of Gal4-VP16, Gal4-VP64, tetR-VP64, ZFHD1-VP64, Gal4-KRAB, and HAP1-VP16.

In some embodiments, the chimeric receptor provided herein further comprises a signal sequence, a detectable label, a tumor-specific cleavage site, a disease-specific cleavage site, or a combination thereof.

Provided herein, among others, includes a recombinant nucleic acid comprising a nucleotide sequence encoding the chimeric receptor described herein. In some embodiments, the nucleotide sequence is incorporated into an expression cassette or an expression vector. In some embodiments, the expression vector is a viral vector. In some embodiments, the viral vector is a lentiviral vector, an adeno virus vector, an adeno-associated virus vector, or a retroviral vector.

Also provided herein includes a recombinant cell comprising the chimeric receptor and/or the recombinant nucleic acid described herein. In some embodiments, the recombinant cell is a eukaryotic cell. In some embodiments, the eukaryotic cell is a mammalian cell. In some embodiments, the mammalian cell is an immune cell, a neuron, an epithelial cell, and endothelial cell, or a stem cell. In some embodiments, the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

In some embodiments, the recombinant cell provided herein comprises: a) a first chimeric receptor and a second chimeric receptor described herein; and/or b) a first nucleic acid and a second nucleic acid described herein. In some embodiments, the first chimeric receptor and the second chimeric receptor do not have the same sequence. In other embodiments, the first nucleic acid or the second nucleic acid do not have the same sequence. In some embodiments, the first chimeric receptor modulates the expression and/or activity of the second chimeric receptor.

In some embodiments, the recombinant cell provided herein further comprises an expression cassette encoding a protein operably linked to a promoter, wherein expression of the protein is modulated by the transcriptional regulator. In some embodiments, the protein is heterologous to the cell. In certain exemplary embodiments, the promoter is a yeast GAL4 promoter. In some embodiments, the protein is a cytokine, a cytotoxin, a chemokine, an immunomodulator, a pro-apoptotic factor, an anti-apoptotic factor, a hormone, a differentiation factor, a de-differentiation factor, an immune cell receptor (e.g., a TCR or CAR), or a reporter.

The present disclosure also provides a cell culture comprising at least one recombinant cell according to the disclosure, and a culture medium.

Additionally, the present disclosure provides a method for making the recombinant cell provided herein. In some embodiments, the method comprises: a) providing a cell capable of protein expression; and b) contacting the provided cell with a recombinant nucleic acid disclosed herein into the provided cell. In some embodiments, the cell is obtained by leukapheresis performed on a sample obtained from a subject, and the cell is contacted ex vivo. In some embodiments, the recombinant nucleic acid is encapsulated in a viral capsid or a lipid nanoparticle.

The present disclosure also provides a pharmaceutical composition comprising a pharmaceutically acceptable carrier, and one or more of the recombinant nucleic acid of the present disclosure. In other embodiments, the present disclosure also provides a pharmaceutical composition comprising a pharmaceutically acceptable carrier, and one or more of the recombinant cell of the present disclosure. In some embodiments, the composition comprises a recombinant nucleic acid of the present disclosure and a pharmaceutically acceptable carrier. In some embodiments, the recombinant nucleic acid is encapsulated in a viral capsid or a lipid nanoparticle.

The present disclosure further provides a system for modulating an activity of a cell, inhibiting a target cancer cell, or treating a health condition in an individual in need thereof. In some embodiments, the system comprises a chimeric receptor of the present disclosure. In other embodiments, the system comprises a recombinant nucleic acid of the present disclosure. In some embodiments, the system comprises a recombinant cell of the present disclosure. In other embodiments, the system comprises a pharmaceutical composition of the present disclosure.

Furthermore, the present disclosure provides a method for modulating an activity of a cell, the method comprising: a) providing a recombinant cell of the present disclosure; and b) contacting the recombinant cell with a selected ligand, wherein binding of the selected ligand to the extracellular ligand-binding domain induces cleavage of a ligand-inducible proteolytic cleavage site and releases the transcriptional regulator, wherein the released transcriptional regulator modulates an activity of the recombinant cell. In some embodiments, the contacting is carried out in vivo, ex vivo, or in vitro.

In some embodiments, the activity of the cell to be modulated is selected from the group consisting of: expression of a gene of interest, cytolytic activity, proliferation, migration, secretion of a molecule, cellular adhesion, differentiation, dedifferentiation, apoptosis, and non-apoptotic death. In some embodiments, the released transcriptional regulator modulates expression of a gene product of the cell. In some embodiments, the released transcriptional regulator modulates expression of a heterologous gene product. In some embodiments, the gene product of the cell is selected from the group consisting of a chimeric antigen receptor, a transcriptional regulator, a transcriptional activator, a transcriptional repressor, a translational regulator, a translational activator, a translational repressor, an activating immuno-receptor, an antibody, a proliferation inducer, a receptor, chemokine, a chemokine receptor, a cytokine, a cytokine receptor, a differentiation factor, a growth factor, a growth factor receptor, a hormone, a metabolic enzyme, a pathogen-derived protein, an RNA guided nuclease, a site-specific nuclease, a T cell receptor, a toxin, a toxin derived protein, an apoptosis inhibitor, an apoptosis inducer, an engineered T cell receptor, an immuno-activator, an immuno-inhibitor, and an inhibiting immuno-receptor. In some embodiments, the released transcriptional regulator modulates differentiation of the cell, and wherein the cell is an immune cell, a stem cell, a progenitor cell, or a precursor cell.

The present disclosure also provides a method for inhibiting an activity of a target cell in an individual, the method comprising administering to the individual an effective number of the recombinant cells provided herein, wherein the recombinant cells inhibit an activity of the target cell in the individual.

In some embodiments, the target cell is a pathogenic cell. In some exemplary embodiments, the pathogenic cell is a cancer cell. In other embodiments, the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-Cell lymphoma cell, a renal cancer cell, a sarcoma cell, a stomach cancer cell, a carcinoma cell, a mesothelioma cell, or a sarcoma cell.

In another aspect, the present disclosure provides a method for the treatment of a health condition in an individual in need thereof. In some embodiments, the method comprises administering to the individual a first therapy comprising an effective number of the recombinant cell provided herein, wherein the recombinant cell treats the health condition in the individual.

In some embodiments, the method for the treatment of a health condition in an individual in need thereof further comprises administering to the individual a second therapy. In some embodiments, the second therapy is selected from the group consisting of chemotherapy, radiotherapy, immunotherapy, hormonal therapy, and toxin therapy. In some embodiments, the first therapy and the second therapy are administered together in the same composition or in separate compositions. In some embodiments, the first therapy and the second therapy are administered at the same time. In some embodiments, the first therapy and the second therapy are administered sequentially. In certain embodiments, the first therapy is administered before the second therapy. In certain embodiments, the first therapy is administered after the second therapy. In other embodiments, the first therapy and the second therapy are administered in rotation.

Among others, the present disclosure also includes a use of one or more of the following for the treatment of a health condition: a) a chimeric receptor provided herein; b) a recombinant nucleic acid provided herein; c) a recombinant cell provided herein; and d) a composition provided herein. In some embodiments, the present disclosure relates to the use of the any of the forgoing for the manufacture of a medicament for the treatment of a health condition. In some embodiments, the health condition is cancer. In certain embodiments, the cancer is a solid tumor, a soft tissue tumor, or a metastatic lesion.

### BRIEF DESCRIPTION OF THE DRAWINGS

FIG. 1A: Schematic depictions of hybrid SynNotch receptors containing anti-GFP (Lag-17) specific extracellular domains, Notch domains, and intracellular signaling domains (i.e. co-stimulatory domains derived from 4-BB and CD3 Zeta.

FIG. 1B: Primary human CD8+ T cells were engineered with the αGFP hybrid SynNotch receptor. The primary T cells must first recognize its cognate antigen (GFP) via their hybrid SynNotch receptor in order to initiate membrane-proximal signaling via kinase cascades as well as release its receptor tethered transcription factor to regulate a user-defined transcriptional gene.

FIG. 1C: Primary human CD8+ T cells expressing the αGFP hybrid SynNotch receptor were co-cultured with K562 expressing either GFP or no GFP for 24 hours. The hybrid SynNotch receptor's ability to regulate user-defined transcription was measured by quantifying the % T cells that expressed the mCherry reporter when primary T cells were co-cultured with K562.

FIG. 1D: Primary human CD8+ T cells expressing the αGFP hybrid SynNotch receptor were co-cultured with K562 expressing either GFP or no GFP. Relative K562 survival over 24 hours was quantified and showed a cytolytic capacity of the hybrid SynNotch receptor.

FIG. 2A: schematic depictions of hybrid SynNotch receptors containing anti-GFP (Lag-17) specific extracellular domains, Notch domains, and signaling domains with CD28 costimulatory domain and 4-1BB co-stimulatory domain.

FIG. 2B: Primary human CD8+ T cells were engineered with the αGFP hybrid SynNotch receptor. The primary T cells must first recognize its cognate antigen (GFP) via their hybrid SynNotch receptor in order to initiate costimulatory signal as well as release its receptor tethered transcription factor to regulate a user-defined transcriptional gene.

FIG. 2C: Primary human CD8+ T cells expressing the αGFP hybrid SynNotch receptor were co-cultured with K562 expressing either GFP or no GFP for 72 hours. The hybrid SynNotch receptor's ability to regulate user-defined transcription was measured by quantifying the % T cells that expressed the mCherry reporter when primary T cells were co-cultured with K562.

FIG. 2D: Primary human CD8+ T cells expressing the αGFP hybrid SynNotch receptor were co-cultured with K562 expressing either GFP or no GFP for 72 hours. The costimulatory signal activity was measured by staining for the upregulation of CD25.

### DETAILED DESCRIPTION OF THE DISCLOSURE

The present disclosure relates generally to a new class of chimeric receptors designed to combine fast time-scale intracellular signal transduction and long time-scale transcription regulation. In particular, some embodiments of the disclosure provides exemplary chimeric receptors (referred herein as “hybrid SynNotch CARs” or “hybrid SynNotch receptors”) that incorporate costimulatory domains and the cytoplasmic tail of the CD3zeta chain in addition to the transcription factor. The architecture of the cytoplasmic tail of these new receptors (e.g., costimulatory domain, CD3zeta, transcription factor) can be configured in multiple ways. Further, the present disclosure identifies hybrid receptor architectures that reliably induce proximal T-cell receptor costimulatory signals and gene regulation in primary human T cells. The new hybrid SynNotch CARs provided herein can simultaneously stimulate short time-scale (e.g., from seconds to minutes) proximal signaling and long-time scale transcriptional regulation that usually takes hours to induce to sufficient levels to observe cellular state changes. The present disclosure demonstrates that, across multiple variations of the transcriptional factors and the signaling domains, the hybrid SynNotch CARs provided herein are able to regulate a user-defined transcriptional circuit in an antigen specific manner. The present disclosure further demonstrates that, across multiple variations between the transcriptional factors and the signaling domains, the hybrid SynNotch CARs provided herein are able to regulate T cell activation and cell cytotoxicity in an antigen specific manner.

Although various features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In addition, the materials, methods, and examples are illustrative only and not intended to be limiting.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration

The term “heterologous” refers to a polypeptide sequence or domain which is not native to a flanking sequence, e.g., wherein the heterologous sequence is not found in nature coupled to the polypeptide sequences occurring at one or both ends.

The term “derived from” as used herein in reference to a protein or polypeptide refers to an origin or source, and may include naturally occurring, recombinant, unpurified or purified polypeptide that is obtained from, is obtained based on a source or original protein or polypeptide. As such, a protein or polypeptide derived from an original protein or polypeptide may include the original protein or polypeptide, in part or in whole, and may be a fragment or variant of the original protein or polypeptide. In some instance, the polypeptide sequence or domain that is derived from a source or origin can be genetically or chemically modified.

The terms “host cell” and “recombinant cell” are used interchangeably herein. It is understood that such terms, as well as “cell”, “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the originally cell or cell line.

As used herein, the term “construct” is intended to mean any recombinant nucleic acid molecule such as an expression cassette, plasmid, cosmid, virus, autonomously replicating polynucleotide molecule, phage, or linear or circular, single-stranded or double-stranded, DNA or RNA polynucleotide molecule, derived from any source, either capable of genomic integration or autonomous replication, including a nucleic acid molecule where one or more nucleic acid sequences has been linked in a functionally operative manner, e.g., operably linked.

The term “operably linked”,” as used herein, denotes a physical or functional linkage between two or more elements, e.g., polypeptide sequences or polynucleotide sequences, which permits them to operate in their intended fashion.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI website at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant cells, proteins, and nucleic acids include cells, proteins, and nucleic acids produced by laboratory methods. Recombinant proteins can include amino acid residues not found within the native (non-recombinant or wild-type) form of the protein or can be include amino acid residues that have been modified, e.g., labeled. The term recombinant can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, and unless otherwise specified, a “therapeutically effective amount” of an agent is an amount sufficient to provide a therapeutic benefit in the treatment or management of a health condition, such as a disease (e.g., a cancer), or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” is an individual under the care of a physician. Thus, the subject can be a human individual or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

All ranges disclosed herein also encompass any and all possible sub-ranges and combinations of sub-ranges thereof Δny listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

One skilled in the art will understand that the chimeric receptors disclosed herein provide signals having a range of characteristics, from low to high ligand-induced transduction and (independently) low to moderate non-induced signal transduction. This range of activities is a new feature that can be exploited to enhance and tune the actions of engineered cells. Further, as described in greater detail below, a number of the receptor variants disclosed herein exhibit improved expression compared to existing SynNotch receptors.

### Notch Receptors

Notch receptors are large transmembrane proteins that normally communicate signals upon binding to surface-bound ligands expressed on adjacent cells. Notch signals rely on cell-to-cell communication, e.g., communication between two contacting cells, in which one contacting cell is a “receiver” cell and the other contacting cell is a “sender” cell. Notch receptors expressed in a receiver cell recognize their ligands (the delta/serrate/lag, or “DSL” family of proteins) expressed on a sending cell. The engagement of notch and delta on these contacting cells leads to a two-step proteolysis of the notch receptor, which ultimately causes the release of the intracellular portion of the receptor (“ICD”) from the membrane into the cytoplasm. Notch has a matrix metalloprotease cleavage site (denoted “S2”), which, when the receptor is not activated is protected from cleavage by the Notch negative regulatory region (“NRR”). The NRR consists of three LIN-12-Notch repeat (“LNR”) modules and a heterodimerization domain (“HD”). It is believed that this proteolysis is regulated by the force exerted by the sending cell: the DSL ligand pulls on the Notch receptor, which changes the conformation of the NRR and exposes the metalloprotease site. This is cleaved by a constitutively active protease (such as ADAM10), which releases the extracellular binding portion and negative regulatory region of the receptor. Release of the ligand binding portion of the receptor in turn exposes another cleavage site (denoted “S3”), which is cleaved by γ-secretase within the cell membrane: this cleavage releases the nuclear homing ICD from the cell membrane. W. R. Gordon et al., Dev Cell (2015) 33:729-36. This released domain alters receiver cell behavior by regulating transcription. Evolutionary divergence of vertebrates and invertebrates was accompanied by at least two rounds of gene duplication in the Notch lineage: flies possess a single Notch gene, worms two (GLP-1 and LIN-12), and mammals four (NOTCH1-4). Transduction of Notch signals relies on three key events: (i) ligand recognition; (ii) conformational exposure of the ligand-dependent cleavage site; and (iii) assembly of nuclear transcriptional activation complexes.

Canonical Notch signals are transduced by a process called regulated intramembrane proteolysis. Notch receptors are normally maintained in a resting, proteolytically resistant conformation on the cell surface, but ligand binding initiates a proteolytic cascade that releases the intracellular domain of the receptor (ICD) from the membrane. The critical, regulated cleavage step is effected by ADAM metalloproteases and occurs at a site called S2 immediately external to the plasma membrane. This truncated receptor, dubbed NEXT (for Notch extracellular truncation), remains membrane-tethered until it is processed at site S3 by γ-secretase, a multiprotein enzyme complex.

After γ-secretase cleavage, the ICD ultimately enters the nucleus, where it nucleates assembly of a transcriptional activation complex that contains a DNA-binding transcription factor, and a transcriptional coactivator of the Mastermind family. This complex then engages one or more additional coactivator proteins such as p300 to recruit the basal transcription machinery and activate the expression of downstream target genes.

Notch receptors have a modular domain organization. The ectodomains of Notch receptors consist of a series of N-terminal epidermal growth factor (EGF)-like repeats that are responsible for ligand binding. O-linked glycosylation of these EGF repeats, including modification by O-fucose, Fringe, and Rumi glycosyltransferases, also modulates the activity of Notch receptors in response to different ligand subtypes in flies and mammals.

The EGF repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the extracellular subunit, and its C-terminal half constitutes the beginning of the transmembrane subunit. Following the extracellular region, the receptor has a transmembrane segment and an intracellular domain (ICD), which includes a transcriptional regulator.

### Compositions of the Disclosure

As described in greater detail below, the present disclosure provides a new class of chimeric polypeptide receptors engineered to combine fast time-scale intracellular signal transduction and long time-scale transcription regulation. In particular, some embodiments of the present disclosure provides, among others, new hybrid SynNotch CAR architectures that incorporate signaling domains (e.g. co-stimulation, CD3zeta, etc.) of CARs that can initiate activation of T cells concomitant with custom transcriptional regulation. In some embodiments, the new hybrid SynNotch CARs provided herein have linear amino acid signaling motif to mediate signaling in T cells added into the cytoplasmic tail of SynNotch receptors. As demonstrated in the Examples and figures, these new receptors can stimulate short time-scale (e.g., from seconds to a minute) proximal signaling as well as long-time scale transcriptional regulation that takes hours to induce to sufficient levels to observe cellular state changes.

### Chimeric Polypeptides

In one aspect, the present disclosure provides chimeric receptor comprising, from N-terminus to C-terminus, one or more of the following components: a) an extracellular ligand-binding domain having a binding affinity for a selected ligand; b) a linking polypeptide comprising a sequence that is at least about 80% sequence identity to a Notch juxtamembrane domain (JMD); c) a transmembrane domain comprising one or more ligand-inducible proteolytic cleavage sites; and d) an intracellular domain comprising, in any order: (i) an intracellular signaling domain (SD) comprising (1) at least one costimulatory domain derived from a signaling molecule and (2) an activation domain, and (ii) a transcriptional regulator. In some embodiments, binding of the selected ligand to the extracellular ligand-binding domain induces cleavage at the ligand-inducible proteolytic cleavage site disposed between the transcriptional regulator and the linking polypeptide. Further, the binding of the selected ligand to the extracellular ligand-binding domain can also induce proximal signaling cascades through the intracellular SD. In some embodiments, the proximal signaling cascades refer to fast time-scale signaling. For instance, the signaling cascades can be induced in seconds to minutes. Alternatively, the signaling cascades can last for seconds to minutes. In some embodiments, such proximal signaling cascades are induced through T-cell receptor costimulatory signals.

### Extracellular Domains and Ligands

In some embodiments, the ECD of the chimeric receptors (e.g., hybrid SynNotch CARs) disclosed herein has a binding affinity for one or more target ligands. The target ligand can be expressed on the surface of a cell, or is otherwise anchored, immobilized, or restrained so that it can exert a mechanical force on the chimeric receptor. The cell can be a pathogen or a human cell. In some embodiments, the human cell can be a tumor cell. In some embodiments, the human cell can be a terminally differentiated cell. As such, without being bound to any particular theory, binding of the ECD of a chimeric receptor provided herein to a cell-surface ligand does not necessarily remove the target ligand from the target cell surface, but instead enacts a mechanical pulling force on the chimeric receptor. For example, an otherwise soluble ligand may be targeted if it is bound to a surface, or to a molecule in the extracellular matrix. In some embodiments, the target ligand is a cell-surface ligand. Non-limiting examples of suitable ligand types include cell surface receptors; adhesion proteins; carbohydrates, lipids, glycolipids, lipoproteins, and lipopolysaccharides that are surface-bound; integrins; mucins; and lectins. In some embodiments, the ligand is a protein. In some embodiments, the ligand is a carbohydrate.

In some embodiments, the ligand is a cluster of differentiation (CD) marker. In some embodiments, the CD marker is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, and MAGE.

In some embodiments, the extracellular domain includes the ligand-binding portion of a receptor. In some embodiments, the extracellular domain includes an antigen-binding moiety that binds to one or more target antigens. In some embodiments, the antigen-binding moiety includes one or more antigen-binding determinants of an antibody or a functional antigen-binding fragment thereof. One skilled in the art upon reading the present disclosure will readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a nanobody, a diabody, a triabody, or a minibody, an F(ab′)₂ fragment, an F(ab) fragment, a single chain variable fragment (scFv), and a single domain antibody (sdAb), or a functional fragment thereof. In some embodiments, the antigen-binding moiety includes an scFv.

The antigen-binding moiety can include naturally-occurring amino acid sequences or can be engineered, designed, or modified to provide desired and/or improved properties such as, e.g., binding affinity. Generally, the binding affinity of an antigen-binding moiety, e.g., an antibody, for a target antigen (e.g., CD19 antigen) can be calculated by the Scatchard method described by Frankel et al., Mol. Immunol, 16:101-06, 1979. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding moiety that does not significantly bind other antigens but binds the antigen with high affinity, e.g., with an equilibrium constant (KD) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

A skilled artisan can select an ECD based on the desired localization or function of a cell that is genetically modified to express a chimeric receptor or hybrid SynNotch CAR of the present disclosure. For example, a chimeric receptor or hybrid SynNotch CAR with an ECD including an antibody specific for a HER2 antigen can target cells to HER2-expressing breast cancer cells. In some embodiments, the ECD of the disclosed hybrid SynNotch CARs is capable of binding a tumor-associated antigen (TAA) or a tumor-specific antigen (TSA). A skill artisan will understand that TAAs include a molecule, such as e.g., protein, present on tumor cells and on normal cells, or on many normal cells, but at much lower concentration than on tumor cells. In contrast, TSAs generally include a molecule, such as e.g., protein which is present on tumor cells but absent from normal cells.

In some cases, the antigen-binding moiety is specific for an epitope present in an antigen that is expressed by a tumor cell, i.e., a tumor-associated antigen. The tumor-associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a myelogenous leukemia cell, a neuroblastoma cell, a glioma, a glioblastoma, a colorectal cancer cell, etc. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

Non-limiting examples of suitable target antigens include CD19, B7H3 (CD276), BCMA (CD269), alkaline phosphatase, placental-like 2 (ALPPL2), green fluorescent protein (GFP), enhanced green fluorescent protein (EGFP), signal regulatory protein α (SIRPα), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD 117), MUC1, NCAM, PAP, PDGFR-0, PRSS21, PSCA, PSMA, ROR1, SSEA-4, TAG72, TEM1/CD248, TEM7R, TSHR, VEGFR2, ALPI, citrullinated vimentin, cMet, and Axl.

In some embodiments, the target antigen is selected from CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, 11Rα, KIT (CD117), MUC1, NCAM, PAP, PDGFR-0, PRSS21, PSCA, PSMA, ROR1, SSEA-4, TAG72, TEM1/CD248, TEM7R, TSHR, VEGFR2, ALPI, citrullinated vimentin, cMet, Axl, GPC2, human epidermal growth factor receptor 2 (Her2/neu), CD276 (B7H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WTi, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, AOC3 (VAP-1), CAM-3001, CCL11 (eotaxin-1), CD125, CD147 (basigin), CD154 (CD40L), CD2, CD20, CD23 (IgE receptor), CD25 (a subunit of the heterodimeric IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGFβ1, TNF-α, VEGF-A, pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin.

Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin 33 (CD61), galactin, K-Ras (V-Ki-ras2 Kirsten rat sarcoma viral oncogene), Ral-B, GPC2, CD276 (B7H3), or IL-13Rα. In some embodiments, the antigen is Her2. In some embodiments, the antigen is ALPPL2. In some embodiments, the antigen is BCMA. In some embodiments, the antigen-binding moiety of the ECD is specific for a reporter protein, such as BFP, GFP, and eGFP. Non-limiting examples of such antigen-binding moiety include a LaG17 anti-GFP nanobody. In some embodiments, the antigen-binding moiety of the ECD includes an anti-BCMA fully-humanized VH domain (FHVH). In some embodiments, the antigen is signal regulatory protein α (SIRPα).

Additional antigens suitable for targeting by the chimeric receptors disclosed herein include, but are not limited to GPC2, human epidermal growth factor receptor 2 (Her2/neu), CD276 (B7H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WTi, HM1B-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional antigens suitable for targeting by the chimeric receptors disclosed herein include, but are not limited to, those associated with an inflammatory disease such as, AOC3 (VAP-1), CAM-3001, CCL11 (eotaxin-1), CD125, CD147 (basigin), CD154 (CD40L), CD2, CD20, CD23 (IgE receptor), CD25 (a subunit of the heteromeric of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, LFA-1 (CD11α), myostatin, OX-40, scleroscin, SOST, TGF®1, TNF-α, and VEGF-A.

Further antigens suitable for targeting by the chimeric receptors and hybrid SynNotch CARs disclosed herein include, but are not limited to the pyruvate kinase isoenzyme type M2 (tumor M2-PK), CD20, CD5, CD7, CD3, TRBC1, TRBC2, BCMA, CD38, CD123, CD93, CD34, CD1a, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is GPC2, CD19, Her2/neu, CD276 (B7H3), IL-13Rα1, or IL-13Rα2. In some embodiments, the antigen is Her2. In some embodiments, the antigen is ALPPL2. In some embodiments, the antigen is BCMA. In some embodiments, the antigen-binding moiety of the ECD is specific for a reporter protein, such as GFP and eGFP. Non-limiting examples of such antigen-binding moiety include a LaG17 anti-GFP nanobody. In some embodiments, the antigen-binding moiety of the ECD includes an anti-BCMA fully-humanized VH domain (FHVH).

In some embodiments, antigens suitable for targeting by the chimeric receptors and hybrid SynNotch CARs disclosed herein include ligands derived from a pathogen. For example, the antigen can be HER2 produced by HER2-positive breast cancer cells. In some embodiments, the antigen can be CD19 that is expressed on B-cell leukemia. In some embodiments, the antigen can be EGFR that is expressed on glioblastoma multiform (GBM) but much less expressed so on healthy CNS tissue. In some embodiments, the antigen can be CEA that is associated with cancer in adults, for example colon cancer.

In some embodiments, the antigen-binding moiety of the ECD is specific for a cell surface target, where non-limiting examples of cell surface targets include CD19, CD30, Her2, CD22, ENPP3, EGFR, CD20, CD52, CD11α, and α-integrin. In some embodiments, the chimeric receptors and hybrid SynNotch CARs disclosed herein include an extracellular domain having an antigen-binding moiety that binds CD19, CEA, HER2, MUC1, CD20, ALPPL2, BCMA, or EGFR. In some embodiments, the chimeric receptors provided herein (e.g., hybrid SynNotch CARs) include an extracellular domain including an antigen-binding moiety that binds CD19. In some embodiments, the chimeric receptors provided herein (e.g., hybrid SynNotch CARs) include an extracellular domain including an antigen-binding moiety that binds ALPPL2. In some embodiments, the chimeric receptors provided herein (e.g., hybrid SynNotch CARs) include an extracellular domain including an antigen-binding moiety that binds BCMA. In some embodiments, the chimeric receptors provided herein (e.g., hybrid SynNotch CARs) include an extracellular domain including an antigen-binding moiety that binds Her2. In some embodiments, the chimeric receptors and hybrid SynNotch CARs disclosed herein include an extracellular domain including an antigen-binding moiety that binds CD19, ALPPL2, BCMA, or Her2.

In some embodiments, the extracellular domain includes an amino acid sequence having at least 70%, at least 75%, at least 80%, at least 85%, at least 90%, at least 95%, at least 96%, at least 97%, at least 98%, at least 99% sequence identity to a sequence set forth in SEQ ID NOS: 3 and 10. In some embodiments, the extracellular domain includes an amino acid sequence having at least 90% sequence identity to a sequence set forth in SEQ ID NOS: 3 and 10. In some embodiments, the extracellular domain includes an amino acid sequence having at least 95% sequence identity to a sequence set forth in SEQ ID NOS: 3 and 10. In some embodiments, the extracellular domain includes an amino acid sequence having 100% sequence identity to a sequence set forth in SEQ ID NOS: 3 and 10. In some embodiments, the extracellular domain includes an amino acid sequence set forth in SEQ ID NOS: 3 and 10, wherein one, two, three, four, or five of the amino acid residues in any one of the SEQ ID NOS: 3 and 10 is/are substituted by a different amino acid residue.

### Linking Polypeptide

As described above, the chimeric receptors of the disclosure include a linking sequence disposed between the extracellular binding domain (ECD) and the transmembrane domain (TMD). SynNotch receptors comprise a heterologous extracellular ligand-binding domain, a linking polypeptide having substantial sequence identity with a Notch receptor JMID including the NRR, a TMD, and an ICD. The negative regulatory region (NRR) of the Notch receptor, which is sandwiched between the ligand binding and transmembrane domains, harbors the structural machinery necessary to maintain metalloprotease resistance in the absence of ligand. The NRR contains three cysteine-rich Lin12/Notch repeats (LNRs) and a “heterodimerization domain” (HD) that contains both the S1 and S2 cleavage sites.

In some embodiments, the linking polypeptide can be derived from a Notch JMD sequence. The Notch JMD sequence may be the sequence from Notch1, Notch2, Notch3, or Notch4, and can be derived from a non-human homolog, such as those from Drosophila, Gallus, Danio, and the like. Four to 50 amino acid residues of the remaining Notch sequence (e.g., 4, 5, 6, 7, 8, 9, 10, 11, 12, 13, 14, 15, 16, 17, 18, 19, 20, or more, amino acid residues) can be used as a polypeptide linker. In some embodiments, the length and amino acid composition of the linker polypeptide sequence are varied to alter the orientation and/or proximity of the ECD and the TMD relative to one another to achieve a desired activity of the chimeric receptor, such as the signal transduction level when ligand induced or in the absence of ligand.

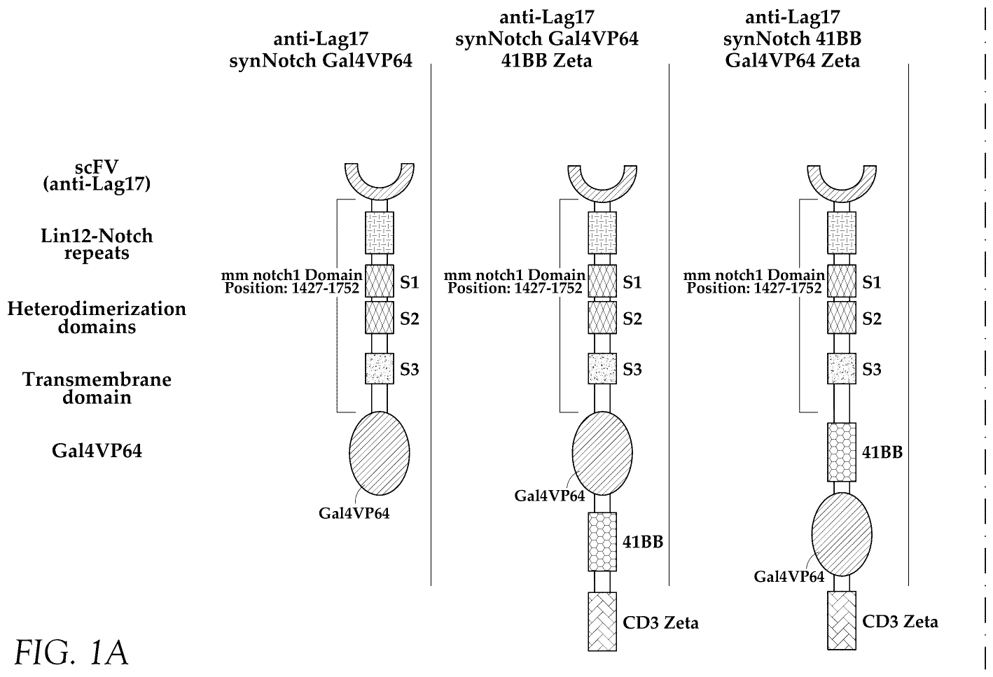

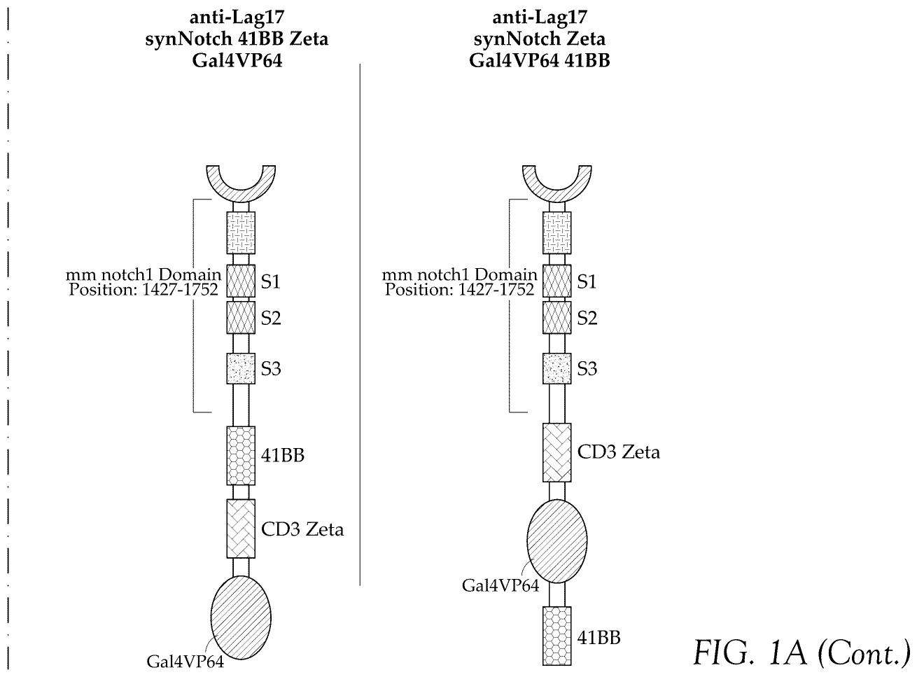

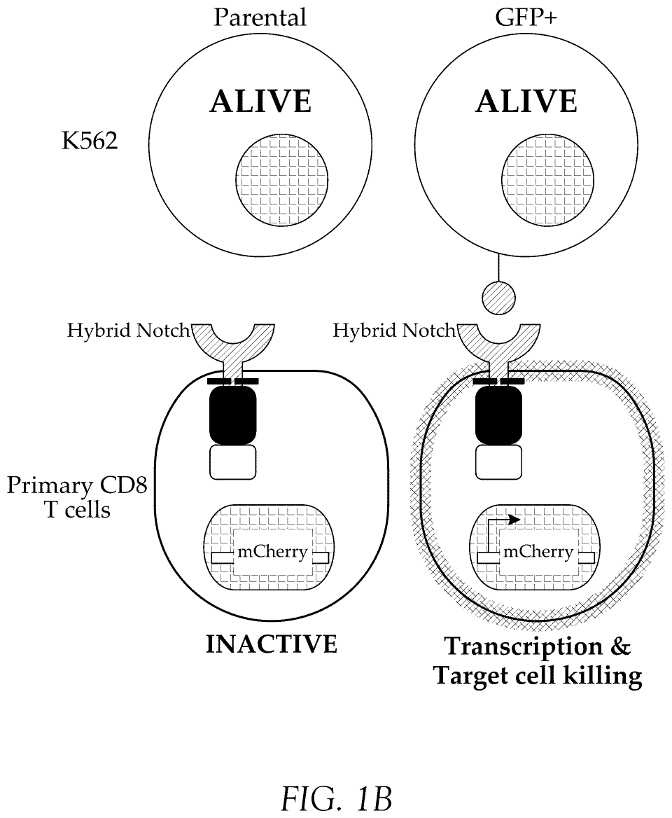

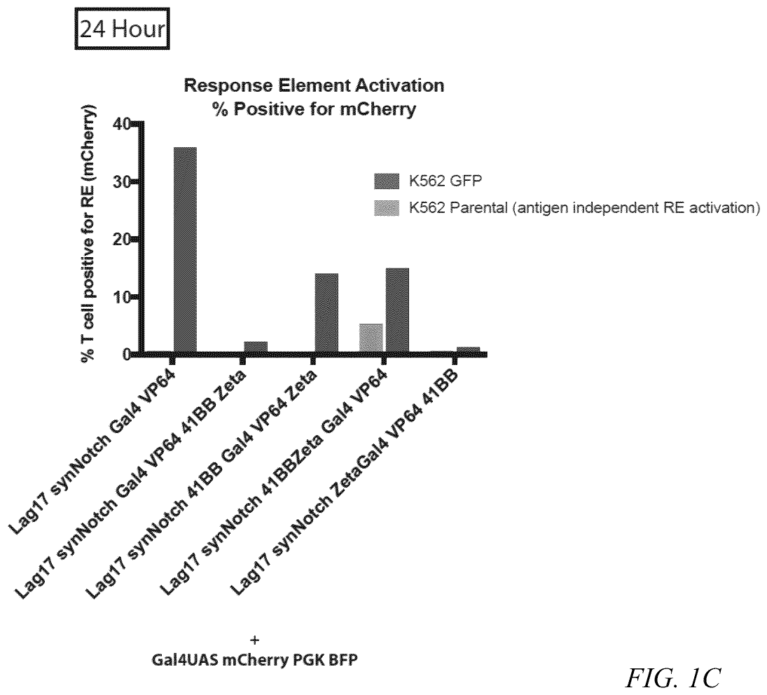

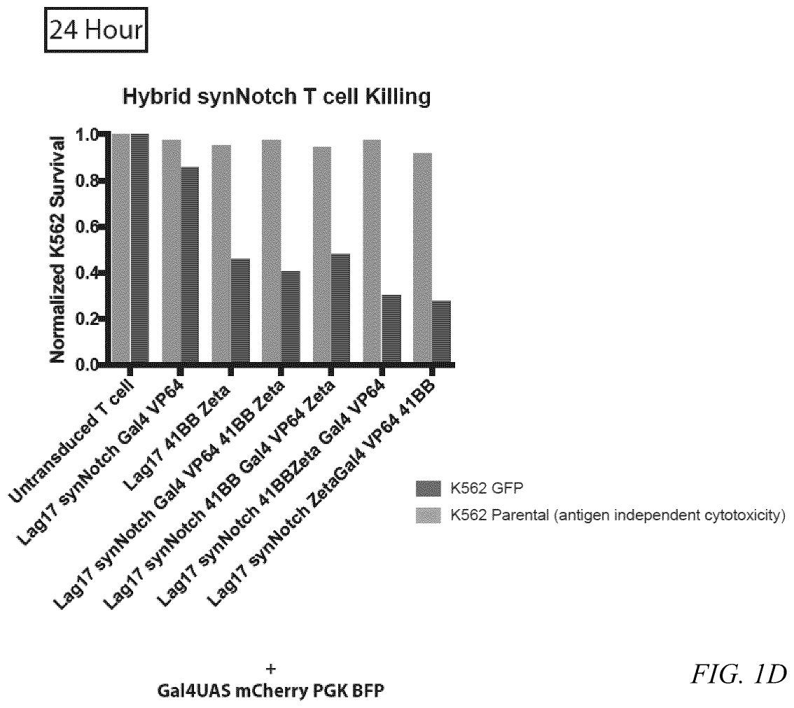

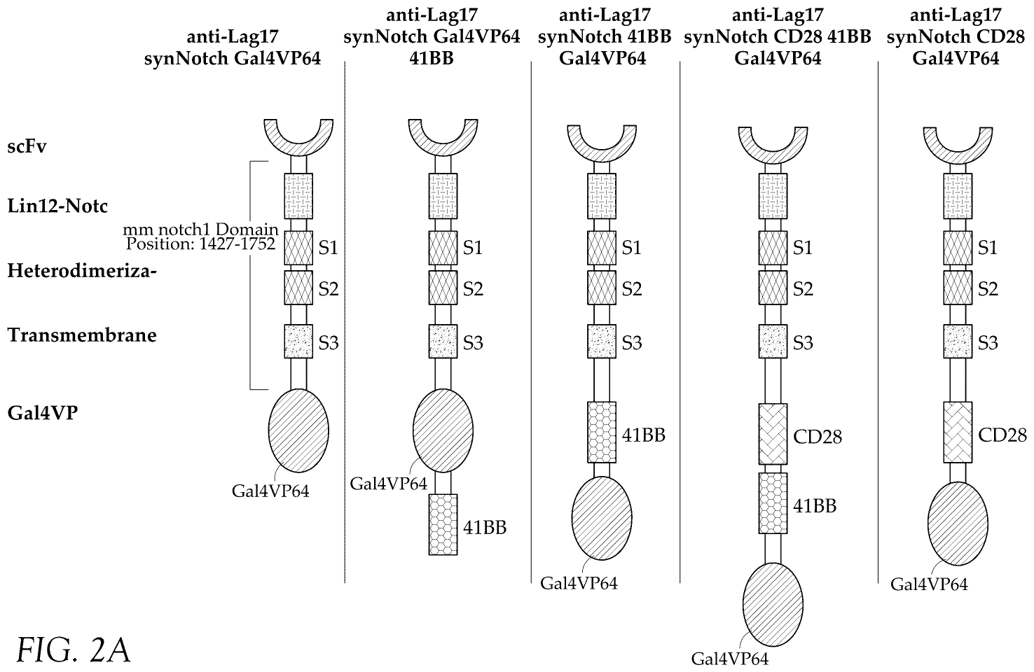

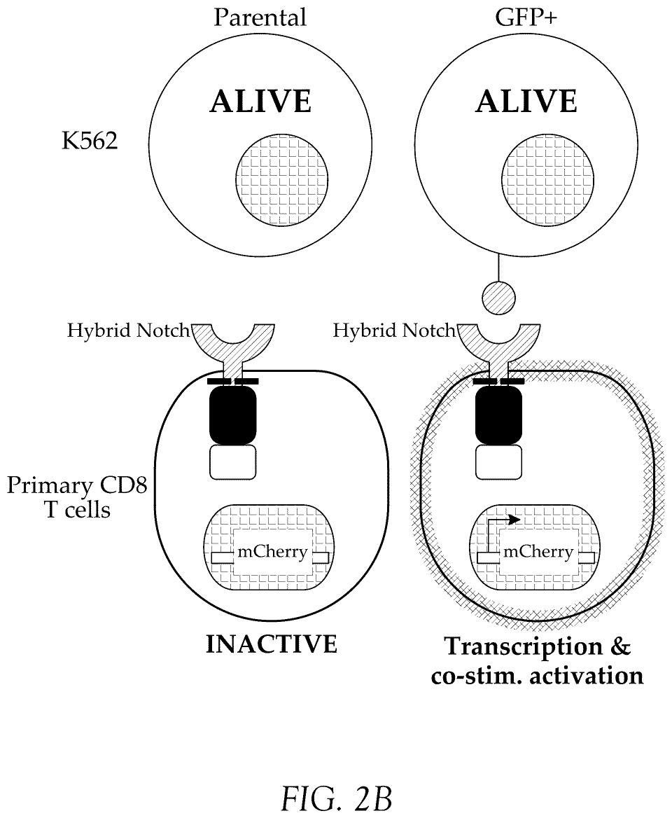

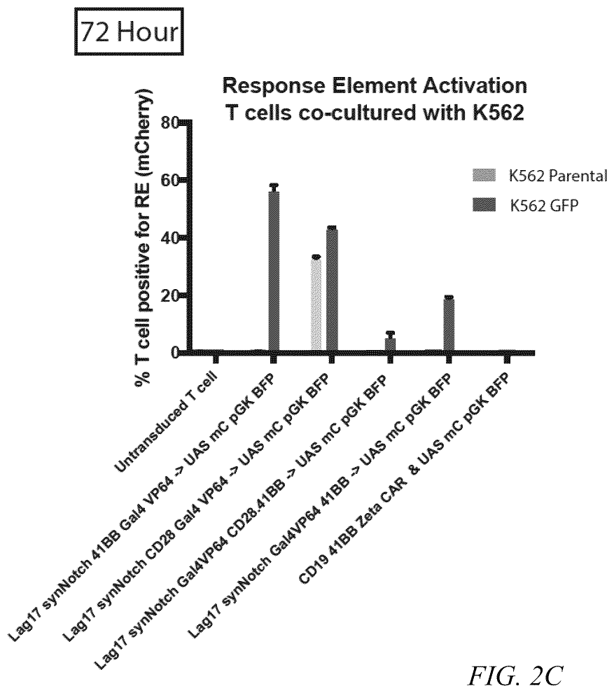

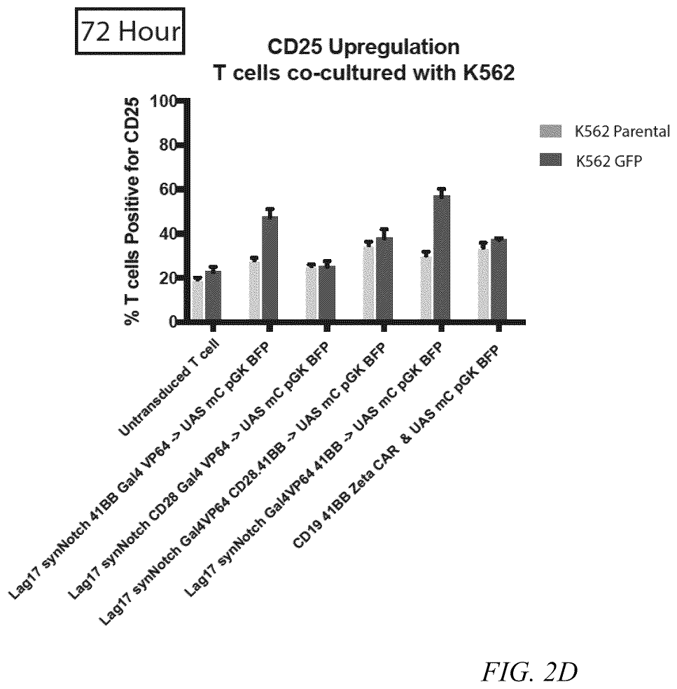

In some embodiments, the linking polypeptide comprises a sequence that is at least about 50%, at least about 60%, at least about 65%, at least about 70%, at least about 75%, at least about 80%, at least about 85%, at least about 90%, or at least about 95% identical to a Notch JMD sequence. In certain embodiments, the linking polypeptide comprises one or more LIN-12-Notch repeat (LNR) of a Notch JMID. In other embodiments, the linking polypeptide further comprises one or more heterodimerization domains (HD) of a Notch JMD. See, for example, FIGS. 1A and 2A.

In some embodiments, the linking polypeptide sequence includes a sequence having at least 80% sequence identity, such as, at least 80%, at least 85%, at least 90%, at least 95%, at least 96%, at least 97%, at least 98%, or 99% sequence identity to a sequence set forth in SEQ ID NO: 11. In some embodiments, the linking polypeptide sequence includes an amino acid sequence having at least 90% sequence identity to a sequence set forth in SEQ ID NO: 11. In some embodiments, the linking polypeptide sequence includes an amino acid sequence having at least 95% sequence identity to a sequence set forth in SEQ ID NO: 11. In some embodiments, the linking polypeptide sequence includes an amino acid sequence having at least 99% sequence identity to a sequence set forth in SEQ ID NO: 11. In some embodiments, the linking polypeptide sequence includes an amino acid sequence identical to a sequence set forth in SEQ ID NO: 11. In some embodiments, the linking polypeptide sequence includes an amino acid sequence set forth in SEQ ID NO: 11, wherein one, two, three, four, or five of the amino acid residues in any one of the SEQ ID NO: 11 is/are substituted by a different amino acid residue.

### Transmembrane Domains

As described above, the chimeric receptors of the disclosure include a TMD comprising one or more ligand-inducible proteolytic cleavage sites.

Examples of proteolytic cleavage sites in a Notch receptor (e.g., S2 or S3) are as described above. Additional proteolytic cleavage sites suitable for the compositions and methods disclosed herein include, but are not limited to, a metalloproteinase cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (SEQ ID NO: 13) (wherein, X represents an arbitrary residue; Hy, a hydrophobic residue such as Leu, Ile, Val, Phe, Trp, Tyr, Val, Met, and Pro), e.g., Pro-X-X-Hy-(Ser/Thr) (SEQ ID NO: 14), e.g., Pro-Leu/Gln-Gly-Met-Thr-Ser (SEQ ID NO: 15) or Pro-Leu/Gln-Gly-Met-Thr (SEQ ID NO: 16). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., Glu-Asn-Leu-Tyr-Thr-Gln-Ser, where the protease cleaves between the glutamine and the serine. Another example of a protease cleavage site that can be included in a proteolytically cleavable linker is an enterokinase cleavage site, e.g., Asp-Asp-Asp-Asp-Lys, where cleavage occurs after the lysine residue. Another example of a protease cleavage site that can be included in a proteolytically cleavable linker is a thrombin cleavage site, e.g., Leu-Val-Pro-Arg. Additional suitable linkers comprising protease cleavage sites include sequences cleavable by the following proteases: a PreScission™ protease (a fusion protein comprising human rhinovirus 3C protease and glutathione-S-transferase), a thrombin, cathepsin B, Epstein-Barr virus protease, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrix metalloproteinase (MT-MMP), stromelysin 3 (or MMP-11), thermolysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the host cell in which the receptor is expressed (for example, TEV) can be used as a further regulatory mechanism, in which activation of the receptor is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

Generally, the TMD suitable for the chimeric receptors disclosed herein can be any transmembrane domain of a Type 1 transmembrane receptor including at least one 7-secretase cleavage site. Detailed description of the structure and function of the 7-secretase complex as well as its substrate proteins, including amyloid precursor protein (APP) and Notch, can, for example, be found in a recent review by Zhang et al., Frontiers Cell Neurosci (2014). Non-limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one 7-secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric receptors of the disclosure is a TMD derived from the TMD of a member of the calsyntenin family, such as, alcadein alpha and alcadein gamma. In some embodiments, the TMD of the chimeric receptors of the disclosure is a TMD known for a Notch receptor. In some embodiments, the TMD of the chimeric receptors of the disclosure is a TMD derived from a different Notch receptor. For example, in a Mini Notch based on human Notch1, the Notch1 TMD can be substituted with a Notch2 TMD, Notch3 TMD, Notch4 TMD, or a Notch TMD from a non-human animal such as Danio rerio, Drosophila melanogaster, Xenopus laevis, or Gallus gallus.

In some embodiments, the amino acid substitution(s) within the TMD includes one or more substitutions within a “GV” motif of the TMD. In some embodiments, at least one of such substitution(s) comprises a substitution to alanine. For example, one, two, three, four, five, or more of the amino acid residues of the transmembrane domain set forth in SEQ ID NO: 12, may be substituted by a different amino acid residue. In some embodiments, the amino acid residue at position 18 and/or 19 of the “GV” motif within SEQ ID NO: 12 is substituted by a different amino acid residue. In some embodiments, the glycine residue at position 18 of SEQ ID NO: 12 is substituted by a different amino acid residue. In some embodiments, the valine residue at position 19 of SEQ ID NO: 12 is substituted by a different amino acid residue. In some embodiments, the transmembrane domain comprises an amino acid sequence having a sequence corresponding to SEQ ID NO: 12 with a mutation at the position corresponding to position 18 of SEQ ID NO: 12, such as G18A mutations. In some embodiments, the transmembrane domain comprises an amino acid sequence having a sequence corresponding to SEQ ID NO: 12 with a mutation at the position corresponding to position 19 of SEQ ID NO: 12, such as V19A mutations. The TMD can be derived from but longer or shorter than SEQ ID NO: 12. For instance, the TMD can be one, two, three, four, or more amino acids longer or shorter than SEQ ID NO: 12. In some embodiments, the TMD includes a sequence having at least 80% sequence identity, such as, at least 80%, at least 85%, at least 90%, at least 95%, at least 96%, at least 97%, at least 98%, or 99% sequence identity to SEQ ID NO: 12.

### Intracellular Domain (ICD)

The chimeric receptors of the disclosure includes an intracellular domain (ICD) comprising, in any order: (i) an intracellular signaling domain (SD) comprising at least one costimulatory domain derived from a signaling molecule and an activation domain, and (ii) a transcriptional regulator. In other words, the ICD of the chimeric receptors of the disclosure can have at least three distinct domains, as depicted in FIGS. 1A and 2A. The three distinct domains can be arranged in specific orders, and can be operably linked to one another via one or more linkers. In some embodiments, as shown in FIG. 1B, the three distinct domains are linked via (GS)n linkers. The n can be any number selected from 1 to 100. For example, the n can be 2, 3, 4, 5, 6, 7, 8, 9, or 10. In other embodiments, the n can be 11, 12, 13, 14, 15, 16, 17, 18, 19, or 20. An exemplary GS linker can have 3 GS repeats and the sequence of GSGSGSGS (SEQ ID NO: 9). One skilled in the art would know how to modify the linker to suit specific uses.

In some embodiments, the intracellular domain of the chimeric receptors of the disclosure further comprises an intracellular signaling domain. The intracellular signaling domain can have at least two distinct domains: at least one costimulatory domain and an activation domain.

In some embodiments, the costimulatory domain comprises a sequence that is derived from a signaling molecule. The signaling molecule can be a protein selected from a class 1 or a class 3 human membrane protein. In some embodiments, the signaling molecule is selected from CD28, 4-1BB, OX40, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-PlA, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC. In some exemplary embodiments, the signaling molecule is selected from the list consisting of OX40, ICOS, 4-1BB, CTLA4, CD28, CD30, CD2, CD27, and CD226, and derivatives, mutants, variants, fragments and combinations thereof. In other embodiments, the signaling molecule is selected from the list consisting of OX40, ICOS, 4-1BB, CTLA4, CD28, CD30, CD2, CD27, and CD226, and derivatives, mutants, variants, fragments and combinations thereof. In some embodiments, the signaling molecule is selected from the group consisting of 4-1BB, BAFF-R, BCMA, BTLA, CD2, CD200R, CD244, CD28, CD300a, CD300f, CD40, CD7, CD72, CD96, CRACC, CRTAM, CTLA4, CXADR, DC-SIGN, GITR, HAVCR2, ICOS, ILT2, ILT3, ILT4, KIR2DL1, KIR3DL1, KLRG1, LAG3, LAIR1, NKG2D, NKR-PlA, NTB-A, PD1, Siglec-3, TACI, TIGIT, TLT-1, and TNR8 (CD30), and derivatives, mutants, variants, fragments and combinations thereof. In other embodiments, the signaling molecule is CD28 or 4-1BB. In one exemplary embodiment, the costimulatory domain comprises a sequence that is derived from CD28. In another exemplary embodiment, the costimulatory domain comprises a sequence that is derived from 4-1BB.

In some embodiments, the activation domain includes one or more includes conserved amino acid motifs that serve as substrates for phosphorylation such as, for example, immunoreceptor tyrosine-based activation motifs (ITAMs). In some embodiments, the activation domain includes at least 1, at least 2, at least 3, at least 4, or at least 5 specific tyrosine-based motifs selected from ITAM motifs, an ITIM motifs, or related intracellular motifs that serve as a substrate for phosphorylation. In some embodiments of the disclosure, the activation domain of the intracellular signaling domain includes at least 1, at least 2, at least 3, at least 4, or at least 5 ITAMs. Generally, any activation domain including an ITAM can be suitably used for the construction of the chimeric receptor s as described herein. An ITAM generally includes a conserved protein motif that is often present in the tail portion of signaling molecules expressed in many immune cells. The motif may include two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways

In some embodiments, the activation domain includes one or more immunoreceptor tyrosine-based activation motifs (ITAMs). In some embodiments, the activation domain is derived from CD3ζ, CD3σ, CD3/, and CD3ε. For instance, in some embodiments, the ITAMs are derived from CD3ζ, CD3σ, CD3/, and CD3ε. In one exemplary embodiment, the ITAM is derived from CD3ζ. In certain embodiments, the ITAM comprises a sequence that is at least about 80, 85, 90, 95, 96, 97, 98, 99, or 100% identical to a CD3ζ ITAM. In some embodiments, the activation domain comprises at least 1, at least 2, at least 3, at least 4, or at least 5 ITAMs independently selected from the ITAMs derived from CD3ζ, FcRγ, and combinations thereof. In some embodiments, the activation domain comprises a CD3ζ ITAM.

In some embodiments, the intracellular domain of the chimeric receptors of the disclosure further comprises a transcriptional regulator. The transcriptional regulator is a biochemical element that acts to activate or repress the transcription of a promoter-driven DNA sequence. Transcriptional regulators suitable for the compositions and methods of the disclosure can be naturally-occurring transcriptional regulators or can be engineered, designed, or modified so as to provide desired and/or improved properties, e.g., modulating transcription. In some embodiments, the transcriptional regulator directly regulates expression of one or more genes involved in differentiation of the cell. In some embodiments, the transcriptional regulator indirectly modulates expression of one or more genes involved in differentiation of the cell by modulating the expression of a second transcription factor which in turn modulates expression of one or more genes involved in differentiation of the cell. It will be understood by a skilled artisan that a transcriptional regulator can be a transcriptional activator or a transcriptional repressor. In some embodiments, the transcriptional regulator is a transcriptional repressor. In some embodiments, the transcriptional regulator is a transcriptional activator. In some embodiments, the transcriptional regulator can further include a nuclear localization signal. In some embodiments, the transcriptional regulator is selected from Gal4-VP16, Gal4-VP64, tetR-VP64, ZFHD1-VP64, Gal4-KRAB, and HAP1-VP16. In some embodiments, the transcriptional regulator is Gal4-VP64.

In some embodiments, the ICD includes a sequence having at least 80% sequence identity, such as, at least 80%, at least 85%, at least 90%, at least 95%, at least 96%, at least 97%, at least 98%, or 99% sequence identity to one or more of SEQ ID NOs: 5-8. In some embodiments, the ICD includes an amino acid sequence having at least 90% sequence identity to one or more of SEQ ID NOs: 5-8. In some embodiments, the ICD includes an amino acid sequence having at least 95% sequence identity to one or more of SEQ ID NOs: 5-8. In some embodiments, the ICD includes an amino acid sequence having at least 100% sequence identity to one or more of SEQ ID NOs: 5-8. In some embodiments, the ICD includes an amino acid sequence of one or more of SEQ ID NOs: 5-8, wherein one, two, three, four, or five of the amino acid residues in one or more of SEQ ID NOs: 5-8 is/are substituted by a different amino acid residue.

### Additional Domains

In some embodiments, the chimeric receptors provided herein can further include an additional region or domain. For example, in some embodiments, the extracellular domains located N-terminally to the TMD can include a membrane localization signal such as a CD8A signal. In other embodiments, the chimeric receptors can include a detectable label, such as a myc tag or His tag, and the like. In additional embodiments, the chimeric receptors provided herein can also include a tumor-specific cleavage site, or a disease-specific cleavage site. In further embodiments, the chimeric receptors provided herein can include a combination of these additional regions.

In some embodiments, the chimeric receptors of the disclosure include one or more of the following components: (a) an extracellular ligand-binding domain having at least 80% sequence identity to any one of SEQ ID NOS: 3 and 10; (b) a linking polypeptide including an amino acid sequence having at least 80% sequence identity to SEQ ID NO: 11; (c) a transmembrane domain including an amino acid sequence having at least 80% sequence identity to SEQ ID NO: 12; and (d) an intracellular domain comprising including one or more amino acid sequences having at least 80% sequence identity to one or more of SEQ ID NOs: 5-8.

In some embodiments, the chimeric receptors of the disclosure include: (a) an extracellular ligand-binding domain having at least 80% sequence identity to any one of SEQ ID NOS: 3 and 10; (b) a linking polypeptide including an amino acid sequence having at least 80% sequence identity to SEQ ID NO: 11; (c) a transmembrane domain including an amino acid sequence having at least 80% sequence identity to SEQ ID NO: 12; and (d) an intracellular domain comprising including three amino acid sequences, each having at least 80% sequence identity to any one of SEQ ID NOs: 5-8, linked by a GS linker. In some embodiments, the GS linker has a sequence set forth in SEQ ID NO: 9.

In some exemplary embodiments, the chimeric receptors of the disclosure includes: (a) an extracellular ligand-binding domain having a sequence set forth in SEQ ID NO: 3 or 10; (b) a linking polypeptide including an amino acid sequence having a sequence set forth in SEQ ID NO: 11; (c) a transmembrane domain including an amino acid sequence having a sequence set forth in SEQ ID NO: 12; and (d) an intracellular domain comprising including three amino acid sequences, each having a sequence set forth in SEQ ID NOs: 5-8, linked by a GS linker. In some embodiments, the GS linker has a sequence set forth in SEQ ID NO: 9. In some embodiments, the chimeric receptors also include a stop-transfer-sequence domain as described herein.

In some embodiments, the chimeric receptors of the disclosure include: (a) an extracellular ligand-binding domain having at least 80% sequence identity to any one of SEQ ID NOS: 3 and 10; (b) a linking polypeptide including an amino acid sequence having at least 80% sequence identity to SEQ ID NO: 11; (c) a transmembrane domain including an amino acid sequence having at least 80% sequence identity to SEQ ID NO: 12; and (d) an intracellular domain comprising including one or more amino acid sequences having at least 80% sequence identity to one or more of SEQ ID NOs: 5-8.

In some embodiments, the chimeric receptor of the disclosure includes an amino acid sequence having at least about 80%, 90%, 95%, 96%, 97, 98%, 99%, or 100% sequence identity to any of the sequences disclosed herein.

### Nucleic Acid Molecules

In another aspect, provided herein are various nucleic acid molecules including nucleotide sequences encoding the chimeric receptors and hybrid SynNotch receptors of the disclosure, including expression cassettes, and expression vectors containing these nucleic acid molecules operably linked to heterologous nucleic acid sequences such as, for example, regulatory sequences which facilitate in vivo expression of the receptor in a host cell.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence encoding a chimeric receptor or hybrid SynNotch receptor including, from N-terminus to C-terminus: a) an extracellular ligand-binding domain having a binding affinity for a selected ligand; b) a linking polypeptide comprising a sequence that is at least about 80% sequence identity to a Notch juxtamembrane domain (JMD); c) a transmembrane domain comprising one or more ligand-inducible proteolytic cleavage sites; and d) an intracellular domain comprising, in any order: (i) an intracellular signaling domain (SD) comprising (1) at least one costimulatory domain derived from a signaling molecule and (2) an activation domain, and (ii) a transcriptional regulator. In some embodiments, binding of the selected ligand to the extracellular ligand-binding domain induces cleavage at the ligand-inducible proteolytic cleavage site disposed between the transcriptional regulator and the linking polypeptide.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the chimeric receptor as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 80%, 90%, 95%, 96%, 97, 98%, 99%, or 100% sequence identity to a chimeric receptor disclosed herein. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOS: 1-45. In some embodiments, the nucleic acid molecules encode a polypeptide with an amino acid sequence having at least about 80%, 90%, 95%, 96%, 97, 98%, 99%, or 100% sequence identity to any one of SEQ ID NOS: 15-31 and 34-45. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOS: 3 and 10. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 3. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 4. In some embodiments, provided herein are nucleic acid molecules encoding a polypeptide with an amino acid sequence having at least about 80%, 90%, 95%, 96%, 97, 98%, 99%, or 100% sequence identity to SEQ ID NO: 5. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOs: 5-8.

The nucleic acid sequences encoding the chimeric receptors can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric receptor or hybrid SynNotch receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acid of the present disclosure can be introduced into a host cell, such as, for example, a human T lymphocyte, to produce a recombinant or engineered cell containing the nucleic acid molecule. Accordingly, some embodiments of the disclosure relate to methods for making a recombinant or engineered cell, including (a) providing a cell capable of protein expression and (b) contacting the provided cell with a recombinant nucleic acid of the disclosure.

Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a subject. In some embodiments, the subject is a human patient.

In some embodiments, the recombinant cell further includes a first and a second nucleic acid molecule as disclosed herein, wherein the first nucleic acid molecule and the second nucleic acid molecule do not have the same sequence. In some embodiments, the recombinant cell further includes a first and a second chimeric receptor or hybrid SynNotch receptor as disclosed herein, wherein the first chimeric receptor or hybrid SynNotch receptor and the second chimeric receptor or hybrid SynNotch receptor do not have the same sequence. In some embodiments, the first chimeric receptor or hybrid SynNotch receptor modulates the expression and/or activity of the second chimeric receptor or hybrid SynNotch receptor.

In some embodiments, the recombinant cell further includes an expression cassette encoding a protein of interest operably linked to a promoter, wherein expression of the protein of interest is modulated by the chimeric receptor transcriptional regulator. Any suitable promoter can be used in connection with the present disclosure. In some embodiments, the promoter comprises a yeast GAL4 promoter. In some embodiments, the protein of interest is heterologous to the recombinant cell. A heterologous protein is one that is not normally found in the cell, e.g., not normally produced by the cell. In principle, there are no particular limitations with regard to suitable proteins whose expression can be modulated by the chimeric receptor transcriptional regulator. Exemplary types of proteins suitable for use with the compositions and methods disclosed herein include cytokines, cytotoxins, chemokines, immunomodulators, pro-apoptotic factors, anti-apoptotic factors, hormones, differentiation factors, dedifferentiation factors, immune cell receptors, or reporters. In some embodiments, the immune cell receptor is a T-cell receptor (TCR). A TCR generally comprises two polypeptides (e.g., polypeptide chains), such as an α-chain of a TCR, a β-chain of a TCR, a γ-chain of a TCR, a δ-chain of a TCR, or a combination thereof. Such polypeptide chains of TCRs and methods of making them are known in the art. In some embodiments, the immune cell receptor is a chimeric antigen receptor (CAR). Generally, a CAR includes an antigen-binding domain, e.g., a single-chain variable fragment (scFv) of an antibody, fused to a transmembrane domain and an intracellular domain. In this case, the antigenic specificity of a CAR can be encoded by a scFv which specifically binds to the antigen, or an epitope thereof. As described in greater detail below, CARs, and methods of making them, are known in the art. In some embodiments, the expression cassette encoding the protein of interest is incorporated into the same nucleic acid molecule that encodes the chimeric receptor of the disclosure. In some embodiments, the expression cassette encoding the protein of interest is incorporated into a second expression vector that is separate from the nucleic acid molecule encoding the chimeric receptor of the disclosure. In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the nucleic acids, and recombinant cells of the disclosure can be incorporated into compositions, including pharmaceutical compositions. Such compositions generally include the nucleic acids, and/or recombinant cells, and a pharmaceutically acceptable excipient, e.g., carrier.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the chimeric receptors and Notch receptors of the disclosure can also be administered by transfection or infection using methods known in the art, including but not limited to the methods described in McCaffrey et al. (Nature 418:6893, 2002), Xia et al. (Nature Biotechnol. 20:1006-10, 2002), or Putnam (Am. J. Health Syst. Pharm. 53:151-60, 1996, erratum at Am. J. Health Syst. Pharm. 53:325, 1996).

### Methods of the Disclosure

Administration of any one of the compositions (e.g., therapeutic compositions) described herein, e.g., nucleic acids, chimeric polypeptides, recombinant cells, and pharmaceutical compositions, can be used to treat patients for relevant health conditions or diseases, such as cancers and chronic infections. In some embodiments, the nucleic acids, recombinant cells, and pharmaceutical compositions described herein can be incorporated into therapeutic agents for use in methods of treating an individual who has, who is suspected of having, or who may be at high risk for developing one or more autoimmune disorders or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Accordingly, in one aspect, some embodiments of the disclosure relate to methods for inhibiting an activity of a target cell in an individual, the methods include administering to the individual a first therapy including one or more of nucleic acids, chimeric polypeptides, recombinant cells, and pharmaceutical compositions as disclosed herein, wherein the first therapy inhibits the target cell. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit an activity of the target cells in the individual. Generally, the target cells of the disclosed methods can be any cell type in an individual and can be, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

In another aspect, some embodiments of the disclosure relate to methods for the treatment of a health condition (e.g., disease) in an individual in need thereof, the methods include administering to the individual a first therapy including one or more of the recombinant cells including a chimeric receptor as disclosed herein, and/or pharmaceutical compositions as disclosed herein, wherein the first therapy treats the health condition in the individual. In some embodiments, the methods include administering to the individual a first therapy including an effective number of the recombinant cells as disclosed herein, wherein the recombinant cells treat the health condition.

In another aspect, some embodiments of the disclosure relate to methods for assisting in the treatment of a health condition (e.g., disease) in an individual in need thereof, the methods including administering to the individual a first therapy including one or more of chimeric polypeptides (e.g., chimeric receptors), nucleic acids, recombinant cells, and pharmaceutical compositions as disclosed herein, and a second therapy, wherein the first and second therapies together treat the disease in the individual. In some embodiments, the methods include administering to the individual a first therapy including an effective number of the recombinant cells as disclosed herein, wherein the recombinant cells treat the health condition.

### Administration of Recombinant Cells to an Individual

In some embodiments, the methods of the disclosure involve administering an effective amount of the recombinants cells of the disclosure to an individual in need of such treatment. This administering step can be accomplished using any method of implantation delivery in the art. For example, the recombinant cells of the disclosure can be infused directly in the individual's bloodstream or otherwise administered to the individual.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the lifetime of the individual, i.e., long-term engraftment.

When provided prophylactically, the recombinant cells described herein can be administered to an individual in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A composition including recombinant cells can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, e.g., via infusion or injection. For example, a population of recombinant cells are administered other than directly into a target site, tissue, or organ, such that it enters, the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments of the disclosed methods, the individual is a mammal. In some embodiments, the mammal is a human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

Methods for CAR design, delivery and expression in T cells, and the manufacturing of clinical-grade CAR-T cell populations are known in the art. See, for example, Lee et al., Clin Cancer Res (2012) 18(10):2780-90, hereby incorporated by reference in its entirety. For example, the engineered CARs may be introduced into T cells using retroviruses, which efficiently and stably integrate a nucleic acid sequence encoding the chimeric antigen receptor into the target cell genome. An exemplary method is described in Example 2 below.

Other methods known in the art include, but are not limited to, lentiviral transduction, transposon-based systems, direct RNA transfection, and CRISPR/Cas systems (e.g., type I, type II, or type III systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, Cas12a (Cpf1), Cas13a (C2c2), Cas13b, Cas13d, CasF, CasG, CasH, Csy1, Csy2, Csy3, Cse1 (or CasA), Cse2 (or CasB), Cse3 (or CasE), CasX, CasY, Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966, etc.).

In some embodiments, a recombinant adeno-associated virus (AAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV requires that the following components are present within a single cell (denoted herein as a packaging cell): a rAAV genome, AAV rep and cap genes separate from (e.g., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. Production of pseudotyped rAAV is disclosed in, for example, international patent application publication number WO 01/83692.

The CAR-T cells, once they have been expanded ex vivo in response to, for example, an autoimmune disease antigen, can be reinfused into the subject in a therapeutically effective amount.

The precise amount of CAR T cells to be administered can be determined by a physician with consideration of individual differences in age, weight, extent of disease and condition of the subject.

Administration of T cell therapies may be defined by number of total cells per infusion or number of cells per kilogram of body weight, especially for pediatric subjects (e.g., patients). As T cells replicate and expand after transfer, the administered cell dose may not resemble the final steady-state number of cells. In some embodiments, a pharmaceutical composition including the CAR T cells of the present disclosure may be administered at a dosage of 10⁴ to 10¹⁰ total cells. In another embodiment, a pharmaceutical composition including the CAR T cells of the present disclosure may be administered at a dosage of 10³ to 10⁸ cells/kg body weight, including all integer values within those ranges.

Compositions including the CAR T cells of the present disclosure may also be administered multiple times at these dosages. The cells can be administered by using infusion techniques that are known in the art (see, for example, Rosenberg et al., New Engl J Med, (1988) 319:1676). The optimal dosage and treatment regimen for a particular subject can be determined by one skilled in the art by monitoring the subject for signs of disease and adjusting the treatment accordingly.

In some embodiments, administration of any of the compositions embodied herein, for the treatment of, for example, an autoimmune or inflammatory disease, can be combined with other cell-based therapies, for example, stem cells, antigen presenting cells, pancreatic islets etc.

The composition of the present disclosure may be prepared in a manner known in the art and in a manner suitable for parenteral administration to mammals, particularly humans, including a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the present disclosure may also include other supplementary physiologically active agents.

The carrier must be pharmaceutically “acceptable” in the sense of being compatible with the other ingredients of the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the CAR-T cells. In general, the compositions are prepared by uniformly and/or intimately bringing into association any active ingredients with liquid carriers.

In some embodiments, the composition is suitable for parenteral administration. In another embodiment, the composition is suitable for intravenous administration.

Compositions suitable for parenteral administration include aqueous and nonaqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

### Additional Therapies

As discussed above, any one of the compositions as disclosed herein, e.g., the chimeric polypeptides (e.g., chimeric receptors), recombinant nucleic acids, recombinant cells, cell cultures, and pharmaceutical compositions described herein can be administered to a subject in need thereof as a single therapy (e.g., monotherapy). In addition or alternatively, in some embodiments of the disclosure, the chimeric polypeptides (e.g., chimeric receptors), recombinant nucleic acids, recombinant cells, cell cultures, and pharmaceutical compositions described herein can be administered to the subject in combination with one or more additional therapies, e.g., at least one, two, three, four, or five additional therapies. Suitable therapies to be administered in combination with the compositions of the disclosure include, but are not limited to chemotherapy, radiotherapy, immunotherapy, hormonal therapy, toxin therapy, targeted therapy, and surgery. Other suitable therapies include therapeutic agents such as chemotherapeutics, anti-cancer agents, and anti-cancer therapies.

Administration “in combination with” one or more additional therapies includes simultaneous (concurrent) and consecutive administration in any order. In some embodiments, the one or more additional therapies is selected from the group consisting of chemotherapy, radiotherapy, immunotherapy, hormonal therapy, toxin therapy, and surgery. The term chemotherapy as used herein encompasses anti-cancer agents. Various classes of anti-cancer agents can be suitably used for the methods disclosed herein. Non-limiting examples of anti-cancer agents include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

The present disclosure also contemplates the combination of the composition of the disclosure with other drugs and/or in addition to other treatment regimens or modalities such as surgery. When the composition of the present disclosure is used in combination with known therapeutic agents the combination may be administered either in sequence (either continuously or broken up by periods of no treatment) or concurrently or as an admixture. In the case of, for example, autoimmune diseases, treatment includes administering to the subject the compositions embodied herein, e.g. autologous T cells transduced or contacted with a CAR embodied herein and one or more anti-inflammatory agents and/or therapeutic agents. The anti-inflammatory agents include one or more antibodies which specifically bind to pro-inflammatory cytokines, e.g., pro-inflammatory cytokines such as IL-1, TNF, IL-6, GM-CSF, and IFN-γ. In some embodiments, the antibodies are anti-TNFα, anti-IL-6 or combinations thereof. In some embodiments, one or more agents, other than antibodies can be administered which decrease pro-inflammatory cytokines, e.g. non-steroidal anti-inflammatory drugs (NSAIDs). Any combination of antibodies and one or more agents can be administered which decrease pro-inflammatory cytokines.

Treatment in combination is also contemplated to encompass the treatment with either the composition of the disclosure followed by a known treatment, or treatment with a known agent followed by treatment with the composition of the disclosure, for example, as maintenance therapy. For example, in the treatment of autoimmune diseases, excessive and prolonged activation of immune cells, such as T and B lymphocytes, and overexpression of the master pro-inflammatory cytokine tumor necrosis factor alpha (TNF), together with other mediators such as interleukin-6 (IL-6), interleukin-1 (IL-1), and interferon gamma (IFN-γ), play a central role in the pathogenesis of autoimmune inflammatory responses in rheumatoid arthritis (RA), inflammatory bowel disease (IBD), Crohn's disease (CD), and ankylosing spondylitis (AS).

Non-steroidal anti-inflammatory drugs (NSAIDs), glucocorticoids, disease-modifying anti-rheumatic drugs (DMARDs) are traditionally used in the treatment of autoimmune inflammatory diseases. NSAIDs and glucocorticoids are effective in the alleviation of pain and inhibition of inflammation, while DMARDs have the capacity of reducing tissue and organ damage caused by inflammatory responses. More recently, treatment for RA and other autoimmune diseases has been revolutionized with the discovery that TNF is critically important in the development of the diseases. Anti-TNF biologics (such as infliximab, adalimumab, etanercept, golimumab, and certolizumab pegol) have markedly improved the outcome of the management of autoimmune inflammatory diseases.

Non-steroidal anti-inflammatory drugs have the analgesic, antipyretic, and anti-inflammatory effect, frequently used for the treatment of conditions like arthritis and headaches. NSAIDs relieve pain through blocking cyclooxygenase (COX) enzymes. COX promotes the production of prostaglandins, a mediator which causes inflammation and pain. Although NSAIDs have different chemical structures, all of them have the similar therapeutic effect, e.g., inhibition of autoimmune inflammatory responses. In general, NSAIDs can be divided into two broad categories: traditional non-selective NSAIDs and selective cyclooxygenase-2 (COX-2) inhibitors (For a review, see, P. Li et al., Front Pharmacol (2017) 8:460).

In addition to anti-TNF agents, the biologics targeting other pro-inflammatory cytokines or immune competent molecules have also been extensively studied and actively developed. For example, abatacept, a fully humanized fusion protein of extracellular domain of CTLA-4 and Fc fraction of IgG1, has been approved for the RA patients with inadequate response to anti-TNF therapy. The major immunological mechanism of abatacept is selective inhibition of co-stimulation pathway (CD80 and CD86) and activation of T cells. Tocilizumab, a humanized anti-IL-6 receptor monoclonal antibody was approved for RA patients intolerant to DMARDs and/or anti-TNF biologics. This therapeutic mAb blocks the transmembrane signaling of IL-6 through binding with soluble and membrane forms of IL-6 receptor. Biological drugs targeting IL-1 (anakinra), Th1 immune responses (IL-12/IL-23, ustekinumab), Th17 immune responses (IL-17, secukinumab) and CD20 (rituximab) have also been approved for the treatment of autoimmune diseases (For a review see, P. Li et al., Front Pharmacol (2017) 8:460).

Accordingly, in some embodiments, the methods of the disclosure include administration of a composition disclosed herein to a subject individually as a single therapy (e.g., monotherapy). In some embodiments, a composition of the disclosure is administered to a subject as a first therapy in combination with a second therapy. In some embodiments, the second therapy is selected from the group consisting of chemotherapy, radiotherapy, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Methods for Modulating an Activity of a Cell

In another aspect, provided herein are various methods for modulating an activity of a cell. The methods include the steps of: (a) providing an effective amount of any of the recombinant cells provided herein, and (b) contacting it with a selected ligand, wherein binding of the selected ligand to the extracellular ligand-binding domain induces cleavage of a ligand-inducible proteolytic cleavage site and releases the intracellular domain comprising the intracellular signaling domain and the transcriptional regulator, wherein the released intracellular signaling domain and the transcriptional regulator modulates an activity of the recombinant cell. One skilled in the art upon reading the present disclosure will appreciate that the disclosed methods can be carried out in vivo, ex vivo, or in vitro.

Non-limiting exemplary cellular activities that can be modulated using the methods provide herein include, but are not limited to, gene expression, proliferation, apoptosis, non-apoptotic death, differentiation, dedifferentiation, migration, secretion of a gene product, cellular adhesion, and cytolytic activity.

In some embodiments, the released transcriptional regulator modulates expression of a gene product of the cell. In some embodiments, the released transcriptional regulator modulates expression of a heterologous gene product in the cell. A heterologous gene product is one that is not normally found in the native cell, e.g., not normally produced by the cell. For example, the cell can be genetically modified with a nucleic acid including a nucleotide sequence encoding the heterologous gene product.

In some embodiments, the heterologous gene product is a secreted gene product. In some embodiments, the heterologous gene product is a cell surface gene product. In some cases, the heterologous gene product is an intracellular gene product. In some embodiments, the released transcriptional regulator simultaneously modulates expression of two or more heterologous gene products in the cell.

In some embodiments, the heterologous gene product in the cell is selected from the group consisting of a chimeric antigen receptor, a transcriptional regulator, a transcriptional activator, a transcriptional repressor, a translational regulator, a translational activator, a translational repressor, an activating immuno-receptor, an antibody, a proliferation inducer, a receptor, chemokine, a chemokine receptor, a cytokine, a cytokine receptor, a differentiation factor, a growth factor, a growth factor receptor, a hormone, a metabolic enzyme, a pathogen-derived protein, an RNA guided nuclease, a site-specific nuclease, a T cell receptor, a toxin, a toxin derived protein, an apoptosis inhibitor, an apoptosis inducer, an engineered T cell receptor, an immuno-activator, an immuno-inhibitor, and an inhibiting immuno-receptor.

In some embodiments, the released transcriptional regulator modulates differentiation of the cell. Exemplary cell type available for the compositions and methods of the present disclosure include, but are not limited to, immune cells, stem cells, progenitor cells, and precursor cells. Accordingly, in some embodiments, the released transcriptional regulator modulates differentiation of a cell, wherein the cell is an immune cell, a stem cell, a progenitor cells, or a precursor cell.

The chimeric receptors of the disclosure provide a higher degree of expression than a standard SynNotch receptor (e.g., a receptor described in PCT Publication No. WO2016138034A1), when using identical binding domains and ICDs. Depending on the ligand/binding domain pair and their affinity, the chimeric receptors of the disclosure can provide expression enhancement of about 10%, about 15%, about 20%, about 25%, about 30%, about 35%, about 40%, about 45%, or about 50% higher than a corresponding SynNotch receptor (e.g., a receptor described in PCT Publication No. WO2016138034A1).

Additionally, the chimeric receptors of the disclosure can provide transcriptional regulation that responds to the degree of T cell activation, independent of ligand binding. For example, when expressed in a T cell, some receptors of the disclosure provide a stronger ligand-induced signal when the T-cell is activated as compared to the ligand-induced signal when the T-cell is not activated. This permits additional flexibility in use, for example in cases where it is desired to enhance or suppress a T cell response when activated despite the absence of the chimeric receptor ligand.

### Systems and Kits

Also provided herein are systems and kits including the chimeric polypeptides (e.g., chimeric receptors), recombinant nucleic acids, recombinant cells, or pharmaceutical compositions provided and described herein as well as written instructions for making and using the same. For example, provided herein, in some embodiments, are systems and/or kits that include one or more of: a chimeric polypeptide (e.g., chimeric receptor) as described herein, a recombinant nucleic acids as described herein, a recombinant cell as described herein, or a pharmaceutical composition as described herein. In some embodiments, the systems and/or kits of the disclosure further include one or more syringes (including pre-filled syringes) and/or catheters (including pre-filled syringes) used to administer one any of the provided polypeptides (e.g., chimeric receptors), recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a health condition (e.g., disease) in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a system or kit can be in separate containers. In some other embodiments, the components of a system or kit can be combined in a single container.

In some embodiments, a system or kit can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

### EXAMPLES

### Example 1: Exemplary SynNotch Hybrid Receptor Design

This Exampled escribes the construction and structure of the exemplary hybrid SynNotch CARs provided herein.

The exemplary SynNotch hybrid receptors described herein were built by fusing the LaG17 (with lower affinity for GFP) to the mouse Notch1 (NM_008714) minimal regulatory region (Ile1427 to Arg1752, also provided in SEQ ID NO: 4). In addition, Gal4 DNA binding domain, VP64 transactivation domain, 4-1BB co-stimulatory domain, CD28 co-stimulatory domain and CD3zeta domain were placed in the intracellular domains of the receptor. All SynNotch hybrid receptors contain an N-terminal CD8a signal peptide (MALPVTALLLPLALLLHAARP, SEQ ID NO: 1) for membrane targeting and a myc-tag (EQKLISEEDL, SEQ ID NO: 2) for suitable determination of surface expression with α-myc A647; see Morsut et al. (Morsut et al., 2016) for receptor SynNotch receptor peptide sequences). The receptors were cloned into a modified pHR′ SIN:CSW vector containing a silencing-prone spleen focus forming virus (SFFV, which is less susceptible to epigenetic silencing) promoter for all primary T cell experiments. The pHR′ SIN:CSW vector was also modified to make the response element plasmids. Five copies of a Gal4 DNA-binding domain's target sequence (GGAGCACTGTCCTCCGAACG, SEQ ID NO: 11) were cloned to a minimal CMV promoter. Also included in the response element plasmids is a 3-phosphoglycerate kinase (PGK) promoter that constitutively drives BFP expression to suitably identify transduced T cells and an inducible mCherry reporter. All constructs were cloned via in-fusion cloning (Clontech #ST0345).

FIGS. 1A and 2A schematically illustrate the structure of chimeric receptors containing anti-GFP (Lag-17) specific extracellular domains, Notch domains which include Lin12-Notch repeats, the heterodimerization domains, and the transmembrane domain), and the intracellular domains include the intracellular signaling domains (i.e., 4-1BB, CD28, and/or CD3zeta, etc.) and the transcriptional regulator Gal4-VP64.

As illustrated in FIGS. 1B and 2B, primary human CD8+ T cells were engineered with the anti-GFP hybrid SynNotch receptors with different costimulatory domains. The primary T cells must first recognize its cognate antigen (GFP) via their hybrid SynNotch receptor in order to initiate membrane-proximal signaling via kinase cascades as well as release its receptor tethered transcription factor to regulate a user-defined transcriptional gene.

### Example 2: General Methods and Experimental Procedures

This Example describes the general methods and experimental procedures used in the present disclosure.

### Primary Human T Cell Isolation and Culture:

CD8+ T cells were isolated from anonymous donor blood after apheresis by negative selection (STEMCELL Technologies #15062 and #15063). Blood was obtained from Blood Centers of the Pacific, as approved by the University Institutional Review Board. T cells were cryopreserved in RPMI-1640 (UCSF cell culture core) with 20% human AB serum (Valley Biomedical, #HP1022) and 10% DMSO. After thawing, T cells were cultured in a human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository).

### Lentiviral Transduction of Human T Cells:

Pantropic VSV-G pseudotyped lentivirus was produced via transfection of Lenti-X 293T cells (Clontech #11131D) with a pHR′ SIN:CSW transgene expression vector and the viral packaging plasmids pCMVdR8.91 and pMD2.G using Fugene HD (Promega #E2312). Primary T cells were thawed the same day and, after 24 hours in culture, were stimulated with Human T-Activator CD3/CD28 Dynabeads (Life Technologies #11131D) at a 1:3 cell:bead ratio. At 48 hours, viral supernatant was harvested and in some assays concentrated using Lenti-X concentrator (Clonetech #631231). The primary T cells were exposed to the virus for 24 hours. On day 4 after T cell stimulation, the Dynabeads were removed, and the T cells were expanded until day 9 when they were rested and could be used in assays. T cells were sorted for assays with a Beckton Dickinson (BD) FACs ARIA Fusion. AND-gate T cells exhibiting basal CAR expression were gated out during sorting.

### Cancer Cell Lines:

The cancer cell lines used were K562 myelogenous leukemia cells (ATCC #CCL-243). K562s were lentivirally transduced to stably express surface GFP (GFP fused to the PDGF transmembrane domain). The surface-GFP peptide sequences can be found in Morsut et al. (Morsut et al., 2016). At 72 hours after transductions, cells were sorted on an Aria Fusion cell sorter (BD Biosciences) on the basis of GFP expression to be 100% GFP and subsequently expanded.

### In Vitro Stimulation of SynNotch Hybrid T Cells:

For all in vitro SynNotch T cell stimulations, 5×10⁴ T cells were co-cultured with 5×10⁴ K562 cells in complete human T cell media. After mixing the T cells and cancer cells in round-bottom 96-well tissue culture plates, the cells were centrifuged for 1 min at 400×g to force interaction of the cells, and the cultures were analyzed at 24-72 hr for activation and specific lysis of target tumor cells. All flow cytometry was performed using BD LSR II or Attune NxT Flow Cytometer and the analysis was performed in FlowJo software (TreeStar).

### Assessment of SynNotch AND-Gate T Cell Cytotoxicity:

CD8+ SynNotch AND-Gate T cells were stimulated for 24-72 hr as described above with target cells expressing the indicated antigens. The level of specific lysis of target cancer cells was determined by comparing the fraction of target cells alive in the culture compared to treatment with non-transduced T cell controls. Cell death was monitored by shifting of the target cells out of the side scatter and forward scatter region normally populated by the target cells.

### CD25 Staining:

CD8+ SynNotch hybrid T cells were stimulated with the indicated cancer cell line as described above and stained with anti-CD25 (BD #560987) to determine if they were upregulated.

### Example 3: Transcriptional Activation Activity of Hybrid SynNotch CARs

This Example describes the results of experiments performed to demonstrate the transcriptional activation activity of various exemplary hybrid SynNotch CARs provided herein.

As shown in FIGS. 1C and 2C, several chimeric receptors (e.g., hybrid SynNotch CARs) comprising the CD28, 4-1BB, and/or CD3zeta intracellular signaling domains were found effective in activating the user-defined transcription of the mCherry reporter when primary T cells were co-cultured with K562.

The hybrid SynNotch CAR's ability to release and regulate a user-defined transcriptional circuit was investigated and demonstrated. As diagrammed in FIGS. 1A and 2A, chimeric polypeptides were designed to include a cleavable Notch polypeptide linked with an intracellular signaling domain and an extracellular ligand-binding domain. When the extracellular domain of a chimeric polypeptide binds to its cognate antigen, the cleavable Notch polypeptide is cleaved releasing the intracellular portion which can then lead to the expression of a transgene operatively linked to a promoter that is responsive to the released intracellular domain. As an exemplification of this transcriptional circuit, a chimeric polypeptide was design with an extracellular domain that binds to green fluorescent protein (GFP) and an intracellular Gal4 transcriptional activator domain fused to various signaling domains that, when released, binds an upstream activation sequence (UAS) operatively linked to a mCherry protein, which serves as an activation reporter when the circuit is engineered into cells. The experimental results presented in FIGS. 1C and 2C demonstrate the relative levels of the cells engineered to express the mCherry reporter in the presence of K562 expressing GFP. As can be observed in FIGS. 1C and 2C, most hybrid synNotch receptor variants tested in these experiments could lead to expression of the mCherry reporter only in the presence of K562 expressing GFP and showed minimal to no expression of the mCherry reporter in the presence of parental K562 cells. As demonstrated across multiple variations of the transcriptional factor and the signaling domain, the hybrid synNotch receptors described herein resulted in their capability to regulate a user-defined transcriptional circuit in an antigen-specific manner.

### Example 4: Cell Killing Activity of the Hybrid SynNotch CARs

This Example describes the results of experiments performed to demonstrate the cell killing activity of various exemplary hybrid SynNotch CARs provided herein.

As shown in FIG. 1D, exemplary chimeric receptors (e.g., the hybrid SynNotch CARs) comprising the 4-1BB and/or CD3zeta intracellular signaling domains were found to be effective in killing K562 cells. Furthermore, as shown in FIG. 2D, the costimulatory domains in the chimeric receptors tested herein were found to be capable of activating T-cells, as demonstrated by CD25 upregulation in these T-cells.

The hybrid SynNotch CAR's ability to activate T cells and induce cytotoxicity in an antigen dependent manner was further investigated. As diagrammed in FIGS. 1A and 2A, chimeric polypeptides were designed that include a cleavable Notch polypeptide linked with an intracellular signaling domain and an extracellular ligand-binding domain. When the extracellular domain of a chimeric polypeptide binds to its cognate antigen the intracellular signaling domain has the capacity to elicit membrane proximal signaling via kinase cascades. As an exemplification of this design, a chimeric polypeptide was constructed with an extracellular domain that binds to green fluorescent protein (GFP) and an intracellular domain that contains a Gal4 transcription activator fused to signaling domains that lead to T cell activation such as 4-1BB, CD28, and/or CD3zeta. As shown in FIGS. 1D and 2D, these engineered T cells demonstrated an ability to upregulate CD25 and induce cytotoxic response in an antigen dependent manner. As can be observed in FIGS. 1D and 2D, most hybrid synNotch receptor variants tested in this experiment led to increased upregulation of CD25 and cytolytic activity only in the presence of K562 expressing GFP. As demonstrated across multiple variations between the transcriptional factor and the signaling domain, the hybrid synNotch receptors described herein resulted in their ability to regulate T cell activation and cytotoxicity in an antigen-specific manner.

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

What is claimed is:

1. A chimeric receptor comprising, from N-terminus to C-terminus:
a) an extracellular ligand-binding domain having a binding affinity for a selected ligand;
b) a linking polypeptide comprising a sequence that is at least about 80% sequence identity to a Notch juxtamembrane domain (JMD);
c) a transmembrane domain comprising one or more ligand-inducible proteolytic cleavage sites; and
d) an intracellular domain comprising, in any order:
(i) an intracellular signaling domain (SD) comprising at least one activation domain, and
(ii) a transcriptional regulator, and
wherein binding of the selected ligand to the extracellular ligand-binding domain induces cleavage at the ligand-inducible proteolytic cleavage site disposed between the transcriptional regulator and the linking polypeptide, and
wherein binding of the selected ligand to the extracellular ligand-binding domain induces proximal signaling cascades through the intracellular SD.

2. The chimeric receptor of claim 1, wherein the extracellular domain comprises an antigen-binding moiety capable of binding to a ligand on the surface of a cell.

3. The chimeric receptor of claim 2, wherein the cell is a pathogen.

4. The chimeric receptor of claim 3, wherein the cell is a human cell.

5. The chimeric receptor of claim 4, wherein the human cell is a tumor cell.

6. The chimeric receptor of claim 4, wherein the human cell is a terminally differentiated cell.

7. The chimeric receptor of any one of claims 1 to 6, wherein the ligand comprises a protein or a carbohydrate.

8. The chimeric receptor of any one of claims 1 to 7, wherein the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), blue fluorescent protein (BFP) enhanced green fluorescent protein (EGFP), and signal regulatory protein α (SIRPα).

9. The chimeric receptor of any one of claims 1 to 8, wherein the ligand is selected from cell surface receptors, adhesion proteins, integrins, mucins, lectins, tumor associated antigens, and tumor-specific antigens.

10. The chimeric receptor of any one of claims 1 to 9, wherein the ligand is a tumor-associated antigen or a tumor-specific antigen.

11. The chimeric receptor of any one of claims 1 to 10, wherein the extracellular ligand-binding domain comprises the ligand-binding portion of a receptor.

12. The chimeric receptor of any one of claims 1 to 11, wherein the antigen-binding moiety is selected from the group consisting of an antibody, a nanobody, a diabody, a triabody, a minibody, an F(ab′)2 fragment, an F(ab)v fragment, a single chain variable fragment (scFv), a single domain antibody (sdAb), and a functional fragment thereof.

13. The chimeric receptor of claim 12, wherein the antigen-binding moiety comprises an scFv.

14. The chimeric receptor of any one of claims 1 to 13, wherein the antigen-binding moiety specifically binds to a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD117), MUC1, NCAM, PAP, PDGFR-0, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

15. The chimeric receptor of claim 14, wherein the tumor-associated antigen is CD19, BCMA, CEA, HER2, MUC1, CD20, ALPPL2, SIRPα, or EGFR.

16. The chimeric receptor of claim 15, wherein the tumor-associated antigen is CD19, BCMA, HER2, or ALPPL2.

17. The chimeric receptor of any one of claims 1 to 16, wherein the linking polypeptide comprises a sequence that is at least about 85%, at least about 90%, or at least about 95% identical to a Notch juxtamembrane domain (JMD).

18. The chimeric receptor of any one of claims 1 to 17, wherein the linking polypeptide comprises one or more LIN-12-Notch repeat (LNR) of a Notch JMID.

19. The chimeric receptor of any one of claims 1 to 18, wherein the linking polypeptide further comprises one or more heterodimerization domains (HD) of a Notch JMD.

20. The chimeric receptor of any one of claims 1 to 19, wherein the linking polypeptide comprises an amino acid sequence having at least 80% sequence identity to SEQ ID NO: 11.

21. The chimeric receptor of any one of claims 1 to 20, wherein the transmembrane domain comprises an amino acid sequence having at least 80% sequence identity to SEQ ID NO: 12.

22. The chimeric receptor of any one of claims 1 to 21, wherein the intracellular SD further comprises at least one costimulatory domain, each derived from a signaling molecule.

23. The chimeric receptor of any one of claims 1 to 22, wherein the signaling molecule comprises a class 1 or a class 3 human membrane protein.

24. The chimeric receptor of any one of claims 1 to 23, wherein the signaling molecule is selected from the group consisting of CD28, 4-1BB, OX40, ICOS, CTLA4, PD1, PD1H, BTLA, B71, B7H1, CD226, CRTAM, TIGIT, CD96, TIM1, TIM2, TIM3, TIM4, CD2, SLAM, 2B4, Ly108, CD84, Ly9, CRACC, BTN1, BTN2, BTN3, LAIR1, LAG3, CD160, CD27, GITR, CD30, TNFR1, TNFR2, HVEM, LT_R, DR3, DCR3, FAS, CD40, RANK, OPG, TRAILR1, TACI, BAFFR, BCMA, TWEAKR, EDAR, XEDAR, RELT, DR6, TROY, NGFR, CD22, SIGLEC-3, SIGLEC-5, SIGLEC-7, KLRG1, NKR-PlA, ILT2, KIR2DL1, KIR3DL1, CD94-NKG2A, CD300b, CD300e, TREM1, TREM2, ILT7, ILT3, ILT4, TLT-1, CD200R, CD300a, CD300f, DC-SIGN, B7-2, Allergin-1, LAT, BLNK, LAYN, SLP76, EMB-LMP1, HIV-NEF, HVS-TIP, HVS-ORF5, and HVS-stpC. In some exemplary embodiments, the signaling molecule is selected from the list consisting of OX40, ICOS, 4-1BB, CTLA4, CD28, CD30, CD2, CD27, and CD226.

25. The chimeric receptor of any one of claims 1 to 24, wherein the signaling molecule is selected from the list consisting of OX40, ICOS, 4-1BB, CTLA4, CD28, CD30, CD2, CD27, and CD226.

26. The chimeric receptor of any one of claims 1 to 25, wherein the activation domain comprises one or more immunoreceptor tyrosine-based activation motifs (ITAMs).

27. The chimeric receptor of any one of claims 1 to 26, wherein the one or more ITAMs are derived from CD3ζ, CD3σ, CD3/, and CD3ε.

28. The chimeric receptor of any one of claims 1 to 27, wherein the one or more ITAMs are at least about 80, 85, 90, 95, 96, 97, 98, 99, or 100% identical to a CD3ζ ITAM.

29. The chimeric receptor of any one of claims 1 to 28, wherein the transcriptional regulator comprises a transcriptional activator, or a transcriptional repressor.

30. The chimeric receptor of any one of the preceding claims 1 to 29, wherein the intracellular domain comprises a nuclear localization sequence and a transcriptional regulator sequence selected from the group consisting of Gal4-VP16, Gal4-VP64, tetR-VP64, ZFHD1-VP64, Gal4-KRAB, and HAP1-VP16.

31. The chimeric receptor of any one of claims 1 to 30, further comprising a signal sequence, a detectable label, a tumor-specific cleavage site, a disease-specific cleavage site, or a combination thereof.

32. A recombinant nucleic acid comprising a nucleotide sequence encoding the chimeric receptor of any one of claims 1 to 31.

33. The recombinant nucleic acid of claim 32, wherein the nucleotide sequence is incorporated into an expression cassette or an expression vector.

34. The recombinant nucleic acid of claim 33, wherein the expression vector is a viral vector.

35. The recombinant nucleic acid of claim 34, wherein the viral vector is a lentiviral vector, an adeno virus vector, an adeno-associated virus vector, or a retroviral vector.

36. A recombinant cell comprising the chimeric receptor according to any one of claims 1 to 31 and/or a recombinant nucleic acid according to any one of claims 32 to 35.

37. The recombinant cell of claim 36, wherein the recombinant cell is a eukaryotic cell.

38. The recombinant cell of claim 37, wherein the eukaryotic cell is a mammalian cell.

39. The recombinant cell of claim 38, wherein the mammalian cell is an immune cell, a neuron, an epithelial cell, and endothelial cell, or a stem cell.

40. The recombinant cell of claim 39, wherein the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

41. The recombinant cell of any one of claims 36 to 40, comprising:
a) a first chimeric receptor and a second chimeric receptor according to any one of claims 1 to 31; and/or
b) a first nucleic acid and a second nucleic acid according to any one of claims 32 to 35;
wherein the first chimeric receptor and the second chimeric receptor do not have the same sequence, and/or the first nucleic acid or the second nucleic acid do not have the same sequence.

42. The recombinant cell of claim 41, wherein the first chimeric receptor modulates the expression and/or activity of the second chimeric receptor.

43. The recombinant cell of any one of claims 36-42, further comprising an expression cassette encoding a protein operably linked to a promoter, wherein expression of the protein is modulated by the transcriptional regulator.

44. The recombinant cell of claim 43, wherein the protein is heterologous to the cell.

45. The recombinant cell of claim 43, wherein the promoter is a yeast GAL4 promoter.

46. The recombinant cell of any one of claims 43-45, wherein the protein is a cytokine, a cytotoxin, a chemokine, an immunomodulator, a pro-apoptotic factor, an anti-apoptotic factor, a hormone, a differentiation factor, a de-differentiation factor, an immune cell receptor (e.g., a TCR or CAR), or a reporter.

47. A cell culture comprising at least one recombinant cell according to any one of claims 36 to 46, and a culture medium.

48. A method for making the recombinant cell according to any one of claims 36 to 46, comprising:
a) providing a cell capable of protein expression;
b) contacting the provided cell with a recombinant nucleic acid according to any one of claims 32 to 35 into the provided cell.

49. The method of claim 48, wherein the cell is obtained by leukapheresis performed on a sample obtained from a subject, and the cell is contacted ex vivo.

50. The method of claim 48, wherein the recombinant nucleic acid is encapsulated in a viral capsid or a lipid nanoparticle.

51. A pharmaceutical composition comprising a pharmaceutically acceptable carrier, and one or more of the following:
a) the recombinant nucleic acid according to any one of claims 32 to 35; and
b) the recombinant cell according to any one of claim 36 or 46.

52. The pharmaceutical composition of claim 48, wherein the composition comprises a recombinant nucleic acid according to any one of claims 32 to 35, and a pharmaceutically acceptable carrier.

53. The pharmaceutical composition of claim 52, wherein the recombinant nucleic acid is encapsulated in a viral capsid or a lipid nanoparticle.

54. A system for modulating an activity of a cell, inhibiting a target cancer cell, or treating a health condition in an individual in need thereof, the system comprising one or more of the following:
a) a chimeric receptor according to any one of claims 1 to 31;
b) a recombinant nucleic acid according to any one of claims 32 to 35;
c) a recombinant cell according to any one of claims 36 to 46; and
d) a pharmaceutical composition according to any one of claims 51 to 53.

55. A method for modulating an activity of a cell, the method comprising:
a) providing a recombinant cell according to any one of claims 36 to 46; and
b) contacting the recombinant cell with a selected ligand, wherein binding of the selected ligand to the extracellular ligand-binding domain induces cleavage of a ligand-inducible proteolytic cleavage site and releases the transcriptional regulator, wherein the released transcriptional regulator modulates an activity of the recombinant cell.

56. The method of claim 55, the contacting is carried out in vivo, ex vivo, or in vitro.

57. The method of any one of claims 55 to 56, wherein the activity of the cell to be modulated is selected from the group consisting of: expression of a gene of interest, cytolytic activity, proliferation, migration, secretion of a molecule, cellular adhesion, differentiation, dedifferentiation, apoptosis, and non-apoptotic death.

58. The method of any one of claims 55 to 57, wherein the released transcriptional regulator modulates expression of a gene product of the cell.

59. The method of any one of claims 55 to 58, wherein the released transcriptional regulator modulates expression of a heterologous gene product.

60. The method of any one of claims 55 to 59, wherein the gene product of the cell is selected from the group consisting of a chimeric antigen receptor, a transcriptional regulator, a transcriptional activator, a transcriptional repressor, a translational regulator, a translational activator, a translational repressor, an activating immuno-receptor, an antibody, a proliferation inducer, a receptor, chemokine, a chemokine receptor, a cytokine, a cytokine receptor, a differentiation factor, a growth factor, a growth factor receptor, a hormone, a metabolic enzyme, a pathogen-derived protein, an RNA guided nuclease, a site-specific nuclease, a T cell receptor, a toxin, a toxin derived protein, an apoptosis inhibitor, an apoptosis inducer, an engineered T cell receptor, an immuno-activator, an immuno-inhibitor, and an inhibiting immuno-receptor.

61. The method of any one of claims 55 to 60, wherein the released transcriptional regulator modulates differentiation of the cell, and wherein the cell is an immune cell, a stem cell, a progenitor cell, or a precursor cell.

62. A method for inhibiting an activity of a target cell in an individual, the method comprising administering to the individual an effective number of the recombinant cells according to any one of claims 36 to 46, wherein the recombinant cells inhibit an activity of the target cell in the individual.

63. The method of claim 62, wherein the target cell is a pathogenic cell.

64. The method of claim 63, wherein the pathogenic cell is a cancer cell.

65. The method of any one of the claims 62-64, wherein the target cell is an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-Cell lymphoma cell, a renal cancer cell, a sarcoma cell, a stomach cancer cell, a carcinoma cell, a mesothelioma cell, or a sarcoma cell.

66. A method for the treatment of a health condition in an individual in need thereof, the method comprising administering to the individual a first therapy comprising an effective number of the recombinant cell according to any one of claims 36 to 46, wherein the recombinant cell treats the health condition in the individual.

67. The method of claim 66, further comprising administering to the individual a second therapy.

68. The method of claim 67, wherein the second therapy is selected from the group consisting of chemotherapy, radiotherapy, immunotherapy, hormonal therapy, and toxin therapy.

69. The method of any one of claims 66 to 68, wherein the first therapy and the second therapy are administered together in the same composition or in separate compositions.

70. The method claim 69, wherein the first therapy and the second therapy are administered at the same time.

71. The method of any one of claims 66 to 70, wherein the first therapy and the second therapy are administered sequentially.

72. The method of claim 71, wherein the first therapy is administered before the second therapy.

73. The method of claim 71, wherein the first therapy is administered after the second therapy.

74. The method of claim 71, wherein the first therapy and the second therapy are administered in rotation.

75. The use of one or more of the following for the treatment of a health condition:
a) a chimeric receptor according to any one of claims 1 to 31;
b) a recombinant nucleic acid according to any one of claims 32 to 35;
c) a recombinant cell according to any one of claims 36 to 46; and
d) a composition according to any one of claims 51 to 53.

76. The use of the invention of any one of the preceding claims for the manufacture of a medicament for the treatment of a health condition.

77. The use of claim 75 or 76, wherein the health condition is cancer.

78. The use of claim 77, wherein the cancer is a solid tumor, a soft tissue tumor, or a metastatic lesion.
